SEQUENCE LISTING (36 sequences)

SEQ ID NO: 1 - protein, 106 aa, Arabidopsis thaliana
MLKDLFQAVSYQNTASLSLFQALSVVESKAMSQTVVLRVAMTCEGCVGAVKRVLGKMEGVESFDVDIKEQKVTVKGNVQPDAVLQTVTKTGKKTAFWEAEGETAKA

SEQ ID NO: 2 - protein, 30 aa, Arabidopsis thaliana
MLKDLFQAVSYQNTASLSLFQALSVVESKA

SEQ ID NO: 3 - protein, 6 aa, Arabidopsis thaliana
  misc_feature (2)..(2): Xaa can be any naturally occurring amino acid
MXCXXC

SEQ ID NO: 4 - protein, 76 aa, Artificial Sequence
  C terminal residues 31-106 sequence
MXXTVXLXVXMXCXGCXGAVXRVLXKXXGVESXDXXXXXXKVXVXGNVXXXAVXXXVXKXGXXTXXWXXXXXXXXX

SEQ ID NO: 5 - protein, 77 aa, Artificial Sequence
  C terminal 31-107 motif
XXXXXXXXVXMXCXGCXGAVXXVLXKXXXXVXXXXXXXXXXXVXVXXXXXXXXXXXXXXKXXXXXXXXXXXXXXXXX

SEQ ID NO: 6 - protein, 76 aa, Arabidopsis thaliana
MSQTVVLRVAMTCEGCVGAVKRVLGKMEGVESFDVDIKEQKVTVKGNVQPDAVLQTVTKTGKKTAFWEAEGETAKA

SEQ ID NO: 7 - protein, 86 aa, Hevea brasiliensis
MSQTVVLKVGMSCEGCVGAVKRVLGKMEGVESYDIDLKEQKVTVKGNVQPEAVLQTVSKTGKKTTFWEAEAPAEPETKPAETVTVA

SEQ ID NO: 8 - protein, 93 aa, Jatropha curcas
MSQTVVLKVGMSCQGCVGAVKRVLGKMEGVESYDIDLQEQKVTVKGNVQPEAVLQTVSKTGKKTEFWEAEAPAAPETKPAETVSEPAETVAVA

SEQ ID NO: 9 - protein, 85 aa, Populus glandulosa
MSQTVVLKVGMSCEGCVGAVKRVLGKMEGVESYDIDLKEQKVTVKGNVQPDAVLQTVSKTGKKTAFWEAEAPAEPAKPAETVAAA

SEQ ID NO: 10 - protein, 84 aa, Populus trichocarpa
MSQTVVLKVGMSCGGCVGAVKRVLGKMEGVESYDIDLKEQKVTVKGNVQPDAVLQTVSKTGKKTTFWEAEAPAEPATAETLAAA

SEQ ID NO: 11 - protein, 84 aa, Zea mays
MAQTVVLKVGMSCEGCVGAVKRVLGKMEGVESYDVDIMEQKVTVKGNVTPDAVLQTVSKTGKKTSFWEAEAVTSESATPAGATA

SEQ ID NO: 12 - protein, 72 aa, Populus trichocarpa
QTVVLKVGMSCEGCVGAVKRVLGKMEGVESYDIDLKEQKVTVKGNVQPDAVLQTVSKTGKKTAFWEAEAPAE

SEQ ID NO: 13 - protein, 81 aa, Oryza sativa
MAETVVLRVGMSCEGCVGAVKRVLGKMQGVESFDVDIKEQKVTVKGNVTPDAVLQTVSKTGKKTSFWDAEPAPVEATAASS

SEQ ID NO: 14 - protein, 79 aa, Medicago truncatula
MSSQTVTLKVGMSCEGCVGAVKRVLGKLDGVESYDIDLKEQKVVVKGNVEPDTVLKTVSKTGKPTAFWEAEAPSETKAQ

SEQ ID NO: 15 - protein, 121 aa, Arabidopsis thaliana
MAQTVVLKVGMSCQGCVGAVNRVLGKMEGVESFDIDIKEQKVTVKGNVEPEAVFQTVSKTGKKTSYWPVEAEAEPKAEADPKVETVTETKTEAETKTEAKVDAKADVEPKAAEAETKPSQV

SEQ ID NO: 16 - protein, 117 aa, Thellungiella halophila
MSQTVVLKVGMSCQGCVGAVNRVLGKMEGVESFDIDIKEQKVTVKGNVEPEAVFQTVSKTGKKTSYWPVDAEAEPKAEAEPKKETETETKTEAETKTEAKVDVEPKLAEAESKPSQV

SEQ ID NO: 17 - protein, 113 aa, Solanum lycopersicum
MSQTVVLKVGMSCQGCVGAVNRVLGKMEGVESFDIDIKEQKVTVIGNVEPEAVFQTVSKTGKKTSYWEEPAPASAPEPETKPVEEKPVEEKPTETPAEPEPKPTEEKPAETVA

SEQ ID NO: 18 - protein, 130 aa, Glycine max
MSSQTVVLKVGMSCQGCAGAVNRVLEKMEGVESFDIDLKEQKVTVKGNVQPDEVLQAVSKSGKKTAFWVDEAQPPENKPSETAPVTSAENDNKASESGPVASENKPPEAAHVASADPETKPSETAVETVA

SEQ ID NO: 19 - protein, 130 aa, Glycine max
MSSQTVVLKVGMSCQGCAGAVNRVLGKMEGVESFDIDLKEQKVTVKGNVESDEVLQAVSKSGKKTAFWVDEAPQSKNKPLESAPVASENKPSEAATVASAEPENKPSEAAIVDSAEPENKPSDTVVETVA

SEQ ID NO: 20 - protein, 126 aa, Medicago truncatula
MSSETVVLKVKMSCSGCSGAVNRVLEKMEGVESFDIDMKEQKVTVKGNVKPQDVFDTVSKTGKKTEFWVEPENNPTETATEAEPENKPSEAVTIDPVEPDNKPSETATVVSIEPENKPSETATVAA

SEQ ID NO: 21 - protein, 108 aa, Plantago major
MSQTVELKVGMSCQGCVGAVKRVLGKMEGVESFDIDIEKQKVTVKGNVEKEAVLQTVSKTGKKTEFWPEEAAEPEAKITEAPAPVEAKPTEAPAAEPESKPTEAVVTA

SEQ ID NO: 22 - protein, 109 aa, Plantago major
MSQTVELKVGMSCQGCVGAVKRVLGKMEGVESFDIDIEKQKVTVKGNVEKEAVLQTVSKTGKKTEFWPEEAAEPEAKITEAPAPVPEAKPTEAPAAEPESKPTEAVVTA

SEQ ID NO: 23 - protein, 75 aa, Knorringia sibirica
MSQTVVLKVEMTCQGCVGAVQRVLGKMEGVESFDVNLEEKKVTVNGNVDPEAVLQKVSKTGRATSFWDESAPPSA

SEQ ID NO: 24 - protein, 75 aa, Rheum australe
MSQTVVLKVEMTCQGCVGAVQRVLGKMEGVESFNVDLKEKKVTVNGNVDPEAVLQKVSKTGKKTSFWDEAAPSSA

SEQ ID NO: 25 - protein, 73 aa, Saccharomonospora cerevisiae
MAEIKHYQFNVVMTCSGCSGAVNKVLTKLEPDVSKIDISLEKQLVDVYTTLPYDFILEKIKKTGKEVRSGYQL

SEQ ID NO: 26 - DNA, 321 bp, Arabidopsis thaliana
atgcttaaagacttgttccaagccgtatcctatcaaaacactgcaagtctctctctctttcaagccttgtcggtggttgaatcgaaagctatgtctcagaccgttgttctcagagtggccatgacatgtgagggatgtgttggagctgtgaaaagagttcttgggaaaatggaaggcgtggagtcatttgacgttgatataaaggagcagaaagtgacggtgaaaggcaacgtgcagccagacgcagttttacagaccgtgacgaaaaccggaaagaaaacggctttttgggaagctgaaggtgaaactgctaaggcttaa

SEQ ID NO: 27 - DNA, 32 bp, Artificial Sequence
  primers FP-CCH-NcoI
aaccatggggatggctcagaccgttgtcctca

SEQ ID NO: 28 - DNA, 31 bp, Artificial Sequence
  primer RP-CCH-PmlI
aacacgtgttaaacttgtgatggcttagtct

SEQ ID NO: 29 - DNA, 30 bp, Artificial Sequence
  primer FP-ATX1-NcoI
aaccatggatgcttaaagacttgttccaag

SEQ ID NO: 30 - DNA, 32 bp, Artificial Sequence
  primer RP-ATX1-PmlI
aacacgtgttaagccttagcagtttcaccttc

SEQ ID NO: 31 - DNA, 20 bp, Artificial Sequence
  AtHMA5 F
tggccagaagcctgtgattt

SEQ ID NO: 32 - DNA, 19 bp, Artificial Sequence
  AtHMA5 R
tggctttcactccctttcc

SEQ ID NO: 33 - DNA, 21 bp, Artificial Sequence
  AtCOPT1..F
gccgttggtttcatgttgttc

SEQ ID NO: 34 - DNA, 19 bp, Artificial Sequence
  AtCOPT1..R
ttttccggtcatcggaggt

SEQ ID NO: 35 - DNA, 22 bp, Artificial Sequence
  AtATC2 F
aggtccaggaatcgttcacaga

SEQ ID NO: 36 - DNA, 22 bp, Artificial Sequence
  AtATC2..R
ccccagctttttaagcctttga

(19) United States
(12) Patent Application Publication
(10) Pub. No.: US 2014/0298534 A1
(43) Pub. Date: Oct. 2, 2014

(54) COPPER RESISTANT PLANT AND USE FOR PHYTOREMEDIATION

(71) Applicant: Academia Sinica, Taipei (TW)

(72) Inventors: Kuo-Chen YEH, TAIPEI (TW); Lung-Jiun SHIN, TAIPEI (TW)

(73) Assignee: Academia Sinica, Taipei (TW)

(21) Appl. No.: 14/106,350

(22) Filed: Dec. 13, 2013

Related U.S. Application Data
(60) Provisional application No. 61/736,926, filed on Dec. 13, 2012.

(51) Int. Cl. C12N 15/82

(52) U.S. Cl. CPC C12N 15/8271
USPC 800/278; 800/298; 800/320.2; 800/320.3; 800/320; 800/320.1; 800/317; 800/312; 800/313; 800/317.3; 800/305; 800/309; 800/307; 800/310

(57) ABSTRACT

The present invention relates to a transgenic plant overexpressing an antioxidant protein 1 (ATX1)-like polypeptide, which exhibits resistance to excess or deficiency of copper. The present invention also relates to a method of phytoremediation of an environment contaminated with copper by growing a transgenic plant overexpressing an ATX1-like polypeptide in the environment.

### CROSS-REFERENCE TO RELATED APPLICATION PARAGRAPH

This application claims the benefit of U.S. Provisional Patent Application No. 61/736,926 filed on Dec. 13, 2012, the content of which is hereby incorporated by reference in its entirety.

### TECHNOLOGY FIELD

The present invention relates to a transgenic plant resistant to copper and its use for phytoremediation.

### BACKGROUND OF THE INVENTION

Copper (Cu) is a transition metal involved in widespread physiological activity, including photosynthesis, mitochondrial respiration, antioxidant activity and ethylene signaling (Puig et al., 2007). Intracellular Cu must be accurately utilized to avoid toxicity caused by the free Cu ion with generated reactive oxygen species (ROS) such as superoxide, hydrogen peroxide and hydroxyl radical that damage proteins, lipids and DNA (Brewer, 2010). Studies of yeast indicate that free Cu ion is restricted to less than one molecule in a cell and is not available for metalloenzymes in physiological activity (Rae et al., 1999). Therefore, Cu uptake must be tightly controlled and Cu must be chelated intracellularly to maintain the homeostasis and delivery.

Plants with a vascular transport system have developed a proficient homeostatic mechanism to manage Cu homeostasis (Puig and Thiele, 2002). A fundamental step in maintaining Cu homeostasis is to control suitable uptake and efflux through the plasma membrane. In yeast and mammals, the transporters responsible for Cu uptake are mainly members of the high-affinity Cu transporter family (Puig and Thiele, 2002). In Arabidopsis, the high-affinity Cu transporters are members of the Cu-transporter protein (COPT) family (Sancenon et al., 2003). COPT1 was the first to be characterized and identified as a Cu uptake transporter (Kampfenkel et al., 1995). COPT1 mRNA accumulates mainly in root tips and is positively regulated by Cu deficiency. Cu acquisition and accumulation in COPT1 knockdown lines is decreased to 50% that of the wild type, and such lines showed a Cu-deficient phenotype (Sancenon et al., 2004). In addition, pollen development is defective in these lines (Sancenon et al., 2004). Thus, COPT1 functions as a major Cu uptake transporter in Arabidopsis and is important for plant growth and development. Recently, COPTS was identified as a prevacuolar compartment/vacuolar Cu exporter. Cu must be released from the root vacuole for long-distance transport to aerial organs (Garcia-Molina et al., 2011; Klaumann et al., 2011; Pilon, 2011).

Heavy metal transporting P-type ATPases (HMAs) 5-8 are also associated with Cu homeostasis (Burkhead et al., 2009). Among the 4 HMAs, HMA5 was reported to be crucial for Cu efflux and vascular translocation (Andres-Colas et al., 2006). HMA5 is mainly expressed in the root and flower and is upregulated by excess Cu. The phenotype of the hma5 mutant is root Cu hypersensitive, and Cu remains in roots under excess Cu conditions (Andres-Colas et al., 2006). The COPT1-knockdown line is sensitive to Cu deficiency, and the hma5 mutant is sensitive to excess Cu. This contrasting Cu-sensitive phenotype between the COPT1 knockdown lines and hma5 mutant supports COPT1 and HMA5 as being responsible for Cu uptake and efflux, respectively. Thus, the balance among the environment, roots and translocation in maintaining suitable intracellular Cu concentration relies on a coordinated expression of COPT1 and HMA5 (Burkhead et al., 2009).

Intracellularly, free Cu must be chelated and delivered to its physiological partner proteins by Cu chaperones after uptake. These Cu chaperones show open-faced β-sandwich global folding with a conserved MXCXXC Cu-binding motif (Harrison et al., 1999). Arabidopsis has at least 3 Cu chaperones, including the Cu chaperone for superoxide dismutase (SOD; CCS) and 2 homologs of yeast antioxidant protein 1 (ATX1), the Cu chaperone (CCH) and ATX1 (Casareno et al., 1998; Chu et al., 2005; Puig et al., 2007). In yeast, CCS is required to transfer Cu to Cu/Zn SOD for the activity (Rae et al., 1999). Arabidopsis has 3 isoforms of Cu/Zn-SOD cytosolic (CSD1), chloroplastic (CSD2) and peroxisomal forms (CSD3)—and only one CCS (Chu et al., 2005). In the ccs mutant, the activities of all 3 Cu/Zn-SOD isoforms are sharply reduced, which indicates that CCS could deliver Cu to CSD2 in the plastid and to CSD1 and CSD3 in the cytosol in Arabidopsis.

CCH was the first Cu chaperone gene identified as a functional homologue of yeast ATX1 and later ATX1 in Arabidopsis (Himelblau et al., 1998; Puig et al., 2007). Both CCH and ATX1 can complement the yeast atx1 mutant (Puig et al., 2007). The analysis of amino acid alignment revealed the conserved Cu-binding motif in these 2 Cu chaperones. However, CCH has a unique C-terminal extension, whereas ATX1 has a probable N-terminal signal peptide (Mira et al., 2001). The C-terminal extension of CCH was proposed to be involved in the translocation of proteins through plasmodesmata to nonnucleated cells, such as sieve elements, to provide a symplastic pathway for Cu redistribution and reutilization (Mira et al., 2001). The mRNA expression of CCH is induced in the absence of Cu and reduced with excess Cu, whereas ATX1 expression is induced by excess Cu. Opposite Cu-regulated expression of CCH and ATX1 suggests that they may function differently in Cu homeostasis in higher plants (Puig et al., 2007). Therefore, more complicated or divergent functions could have evolved for handling different compartmentalization and translocation in higher plants than in yeast.

Previous yeast two-hybrid experiments suggested that full-length ATX1 and C-terminal extension deleted CCH interact with responsive to antagonist 1 (RAN1)/HMA7 and HMA5 (Andres-Colas et al., 2006; Puig et al., 2007). RAN1 possesses Cu-transporting P-type ATPase activity and is required for ethylene signaling in Arabidopsis (Hirayama et al., 1999), whereas HMA5 contributes to Cu efflux (Andres-Colas et al., 2006). Thus, CCH and ATX1 could be involved in Cu homeostasis and ethylene signaling. However, no phenotype related to these functions has been reported. Therefore, the biological importance of CCH and ATX1 in plants remains unknown.

### BRIEF SUMMARY OF THE INVENTION

In this study, we investigated the role of ATX1 and CCH and found a requirement of ATX1 but not CCH for tolerance to excess Cu and Cu deficiency in the vegetative stage of Arabidopsis. In addition, high Cu accumulation and tolerance of ATX1 overexpression lines grown in high Cu soil were also observed. The phenotype of enhanced growth with ATX1 overexpression suggests its positive roles in Cu homeostasis. Furthermore, mutations in the conserved metal binding domain (residues 41-46) and deletion of the N-terminal sequence (residues 1-30) were found to affect the functions of ATX1 (Cu accumulation and resistance to excess or deficiency of Cu).

Therefore, in one aspect, the present invention relates to a transgenic plant transformed with a recombinant polynucleotide comprising a nucleotide sequence encoding an antioxidant protein 1 (ATX1)-like polypeptide, operatively linked to an expression control sequence, wherein the ATX-like polypeptide has an amino acid sequence having at least 30%, 35%, 40%, 45%, 50%, 55%, 60%, 65%, 70%, 75%, 80%, 85%, 90%, or 95% identity to SEQ ID NO: 1 and exhibits a copper binding activity, in which the amino acid sequence of the ATX-like polypeptide has (i) an N-terminal sequence having at least 60%, 65%, 70%, 75%, 80%, 85%, 90%, or 95% identity to SEQ ID NO: 2, corresponding to positions 1 to 30 of SEQ ID NO: 1, and (ii) a C-terminal sequence comprising a copper binding motif of SEQ ID NO: 3, corresponding to positions 41 to 46 of SEQ ID NO: 1.

In another aspect, the present invention relates to a method for producing a transgenic plant, comprising

(a) transforming a plant cell with a recombinant polynucleotide comprising a nucleotide sequence encoding an antioxidant protein 1 (ATX1)-like polypeptide, operatively linked to an expression control sequence, wherein the ATX-like polypeptide has an amino acid sequence having at least 30%, 35%, 40%, 45%, 50%, 55%, 60%, 65%, 70%, 75%, 80%, 85%, 90%, or 95% identity to SEQ ID NO: 1 and exhibits a copper binding activity, in which the amino acid sequence of the ATX-like polypeptide has (i) an N-terminal sequence having at least 60%, 65%, 70%, 75%, 80%, 85%, 90%, or 95% identity to SEQ ID NO: 2, corresponding to positions 1 to 30 of SEQ ID NO: 1, and (ii) a C-terminal sequence comprising a copper binding motif of SEQ ID NO: 3, corresponding to positions 41 to 46 of SEQ ID NO: 1; and

(b) growing the recombinant plant cell obtained in (a) to generate a transgenic plant.

In a further aspect, the present invention relates to a method of phytoremediation of an environment contaminated with copper, comprising:

(a) selecting an environment contaminated with copper; and

(b) growing, in said environment, a transgenic plant transformed with a recombinant polynucleotide comprising a nucleotide sequence encoding an antioxidant protein 1 (ATX1)-like polypeptide, operatively linked to an expression control sequence, wherein the ATX-like polypeptide has an amino acid sequence having at least 30%, 35%, 40%, 45%, 50%, 55%, 60%, 65%, 70%, 75%, 80%, 85%, 90%, or 95% identity to SEQ ID NO: 1 and exhibits a copper binding activity, in which the amino acid sequence of the ATX-like polypeptide has (i) an N-terminal sequence having at least 60%, 65%, 70%, 75%, 80%, 85%, 90%, or 95% identity to SEQ ID NO: 2, corresponding to positions 1 to 30 of SEQ ID NO: 1, and (ii) a C-terminal sequence comprising a copper binding motif of SEQ ID NO: 3, corresponding to positions 41 to 46 of SEQ ID NO: 1, wherein the transgenic plant accumulates and removes an amount of copper from the environment.

In still another aspect, the present invention provides a method for promoting growth a plant, comprising

(a) introducing to a plant cell, a recombinant polynucleotide comprising a nucleotide sequence encoding an antioxidant protein 1 (ATX1)-like polypeptide, operatively linked to an expression control sequence, wherein the ATX-like polypeptide has an amino acid sequence having at least 30%, 35%, 40%, 45%, 50%, 55%, 60%, 65%, 70%, 75%, 80%, 85%, 90%, or 95% identity to SEQ ID NO: 1 and exhibits a copper binding activity, in which the amino acid sequence of the ATX-like polypeptide has (i) an N-terminal sequence having at least 60%, 65%, 70%, 75%, 80%, 85%, 90%, or 95% identity to SEQ ID NO: 2, corresponding to positions 1 to 30 of SEQ ID NO: 1, and (ii) a C-terminal sequence comprising a copper binding motif of SEQ ID NO: 3, corresponding to positions 41 to 46 of SEQ ID NO: 1, to obtain a transformed plant cell, and

(b) producing a transformed plant from said transformed plant, wherein the ATX1-like polypeptide is expressed in the transgenic plant at a level sufficient to promote growth of the plant.

In some embodiments, the N-terminal sequence of the ATX1-like polypeptide is SEQ ID NO: 2.

In some embodiments, the C-terminal sequence comprises SEQ ID NO: 4, corresponding to positions 31 to 106 of SEQ ID NO: 1.

In some embodiments, the C-terminal sequence is selected from the group consisting of SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23 and SEQ ID NO: 24.

In some embodiments, the C-terminal sequence comprises SEQ ID NO: 5.

In some embodiments, the C-terminal sequence is SEQ ID NO: 25.

In one particular embodiment, the ATX1-like protein has the amino acid sequence of SEQ ID NO: 1.

In one particular embodiment, the nucleotide sequence encoding the ATX1-like protein is SEQ ID NO: 26.

In some embodiments, the ATX-like polypeptide is composed of SEQ ID NO: 2 as the N-terminal sequence, fused with the C-terminal sequence selected from the group consisting of SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24 and SEQ ID NO: 25.

In some certain embodiments, the transgenic plant is monocotyledon, including but not limited to rice, barley, wheat, rye, oat, corn, bamboo, sugar cane, onion, leek and ginger.

In some certain embodiments, the transgenic plant is a dicotyledon, including but not limited to Arabidopsis, eggplant, soybean, mung bean, kidney bean, pea, tobacco, lettuce, spinach, sweet potato, carrot, melon, cucumber and pumpkin.

The details of one or more embodiments of the invention are set forth in the description below. Other features or advantages of the present invention will be apparent from the following detailed description of several embodiments, and also from the appending claims.

### BRIEF DESCRIPTION OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 shows CCH and ATX1 T-DNA insertion mutants Salk_{—}138593 (cch) and Salk_{—}026221 (atx1). The cch and atx1 mutants contain a T-DNA inserted (triangle) in the (A) 1st intron of CCH and (B) 1st exon of ATX1. Cyan blue arrows=genomic UTR region. Orange square=genomic exon region. Light green line=genomic intron region. Scale bars indicate 100 bp. (C) The mRNA expression of CCH and AIX1 was examined by RT-PCR. Cycles of PCR are indicated.

FIG. 2 shows growth of wild type and copper chaperone mutants under Cu stress. Plants were grown on half-strength MS agar plates for 14 days. Western blot analysis of protein level of CCH and ATX1 in (A) cch and atx1 mutants and (B) cchatx1 double mutant detected by CCH (α-AtCCH) and ATX1 (α-AtATX1) antibodies. Seeds of wild type and mutants were grown vertically on half-strength MS agar plates and treated with 35 μM CuSO₄ for 17 days. Scale bar=1 cm. (C) Wild-type, cch, atx1 and cchatx1 plants were grown in half-strength MS media and treated with additional Cu as indicated for 17 days, and (D) fresh weight and (E) root length were measured. Data are mean±SD of 4 replicates with 40 seedlings each. **P<0.01 compared with atx1 and cchatx1 in the same condition.

FIG. 3 shows effect of excess Fe, Zn and Cd on wild type and Cu chaperone mutants. Seedlings of wild type and mutants were grown vertically on half-strength MS agar plates with (A) 300 μM FeSO₄, (B) 200 μM ZnSO₄ or (C) 15 μM CdSO₄ for 17 days. Scale bar=1 cm. (D) Cu contents in shoots of treated plants were determined by ICP-OES. Data are mean±SD of 4 replicates with 20 seedlings each.

FIG. 4 shows shoot concentrations of Fe, Zn, Mn, Cu, Mg and Ca in wild type and mutants under Cu stress. Seeds of wild type and mutants were grown vertically on half-strength MS agar plates or treated with an additional 25 or 35 μM CuSO₄ for 17 days. Fe, Zn, Mn, Cu, Mg and Ca contents in shoots were determined by ICP-OES. Data are mean±SD of 4 replicates with 20 seedlings each.

FIG. 5 shows accumulation of CCH and ATX1 protein under excess Cu and Cu deficiency. Plants were grown on half-strength MS phytagel plates for 11 days and transferred to half-strength MS agar plates with 35 μM CuSO₄ for 3 days. Western blot analysis (20 μg total protein per lane) of protein level of (A) CCH and (B) ATX1 detected by CCH (α-AtCCH) and ATX1 (α-AtATX1) antibodies. Commassie blue staining of protein was used to verify the loadings of total protein.

FIG. 6 shows chlorophyll content, lipid peroxidation and antioxidant enzyme activities of Cu chaperone mutants under excess Cu. Plants were grown on half-strength MS phytagel plates for 11 days and transferred to half-strength MS agar plates with additional CuSO₄ as indicated for 3 days. (A) Total chlorophyll and (B) malondialdehyde (MDA) content, and (C) peroxidase (PDX) and (D) catalase (CAT) activity in shoots. Data are mean±SD of 4 replicates with 10 seedlings each. **P<0.01 compared with the wild type in the same condition.

FIG. 7 shows effect of excess Cu on oxidative stress in wild type and Cu chaperone mutants. (A) Shoot carotenoid content, (B) Fv/Fm, (C) root malonaldehyde (MDA) content, and (D) peroxidase (PDX) and (E) catalase (CAT) activity of treated plants described as in FIG. 3. Data are mean±SD of 4 replicates, each containing 10 seedlings. *P<0.05 and **P<0.01 compared with the wild type.

FIG. 8 shows the expression of HMA5 and COPT1 with excess Cu. Plants were grown on half-strength MS phytagel plates for 11 days and transferred to half-strength MS agar plates with additional CuSO₄ as indicated for 3 days. Quantitative PCR analysis of mRNA expression of (A) HMA5 and (B) COPT1 in roots relative to ACT2. Data are mean±SD of 3 replicates with 10 roots each. *P<0.05 compared with the wild type in the same condition.

FIG. 9 shows phenotype of ATX1 transgenic lines, wild type and Cu chaperone mutants with excess Cu. (A) Protein level of ATX1 detected by ATX1 antibody (α-AtATX1) in total protein (20 μg) isolated from each line. Commassie blue staining of protein was used to verify the loadings. Numbers indicate the relative intensity of immunobloting by normalization to the wild type (Wt). (B) Plants were grown on half-strength MS agar plates with 35 μM CuSO₄ for 17 days. Scale bar=1 cm. Shows (C) fresh weight and (D) root length of plants (13-day growth on half-strength MS agar plates with additional CuSO₄ as indicated). Data are mean±SD of 7 replicates with 10 seedlings in (C) and 40 seedlings in (D). Different lowercase letters represent statistical differences by Student t test. Shows representative lines for at least 3 lines of each transgenic construct characterized.

FIG. 10 shows phenotype of CCH and ATX1 transgenic lines, wild type and Cu chaperone mutants with Cu deficiency. (A) Protein level of CCH detected by CCH antibody (α-AtCCH) in total protein (20 μg) isolated from each line. Commassie blue staining was used to verify the loadings. Numbers indicate the relative intensity of immunobloting by normalization to the Wt. (B and C) Plant seeds were grown vertically on half-strength MS agar plates and treated with 10 μM Cu chelator bathocuproine disulfonate (BCS) for 17 days. Scale bar=1 cm. (D) Fresh weight and (E) root length of BCS-treated plants. Shows representatives of at least 3 lines of each transgenic construct characterized. Data are mean±SD of 4 replicates with 10 seedlings each. Different lowercase letters represent statistical differences by Student t test.

FIG. 11 shows the sequence alignment of the MXCXXC motif of Cu chaperones. (A) The sequences of MXCXXC motifs of CCS, CCH and ATX1 in Arabidopsis thaliana (At), Glycine max (Gm), Oryza sativa japonica (Os), Lycopersicon esculentum (Le) and Saccharomyces cerevisiae (Sc). Amino acid residues that are conserved in at least 4 of the 7 proteins are shaded grey, and the identical groups in all 7 proteins are shaded black. The putative amino acid residues of the MXCXXC motif are indicated by a black line. The numbers on the right indicate the last amino acid residues of the protein in alignment. Dashes show gaps in the amino acid sequences for optimized alignment. (B) The relative positions of the MXCXXC motif and 2 C to G substitutions in ATX1 are shown.

FIG. 12 shows phenotype of CG-ATX1 transgenic lines, wild type and atx1 mutants with Cu stress. (A) Protein level of ATX1 detected by ATX1 antibody (α-AtATX1) in total protein (20 μg) isolated from each line. Commassie blue staining was used to verify the loadings. Numbers indicate the relative intensity of immunobloting by normalization to the Wt. nd=not detected. (B) Plant seeds were grown vertically on half-strength MS agar plates for 4 days and then transferred to half-strength MS agar plates with 35 μM CuSO₄ (+Cu) or 10 μM BCS (−Cu) for 13 days. Scale bar=1 cm. (C) Fresh weight and (D) root length of treated plants. Data are mean±SD of 4 replicates with 10 seedlings each. **P<0.01 compared with the wild type in the same condition.

FIG. 13 shows phenotype of ATX1 transgenic lines, wild type and cch mutants in soil with Cu grouting. The seeds of plants were directly grown in soil (A, CK) and 500 μM CuSO₄-presoaked soil (A, +Cu) for 21 days, and grouted with water (CK) or 500 μM CuSO₄ solution (+Cu) 2 times every week, respectively. Scale bar=1 cm. A, lower panel shows the arrangement of plants in soil. (B) Fresh weights of 21-day-old plants with different concentrations of CuSO₄ grouting. (C) The seeds of plants were directly grown in 500 μM CuSO₄-presoaked soil for 21 days, grouted with water 2 times every week. Cu content in shoots was determined by ICP-OES. Data are mean±SD of 4 replicates with 20 seedlings each.

FIG. 14 shows shoot Fe, Zn and Mn concentrations of soil-grown plants. Fe, Zn and Mn contents in shoots of plants described in FIG. 8 were determined by ICP-OES. Data are mean±SD of 4 replicates with 20 seedlings each.

FIG. 15 shows the growth of wild type (Wt) and the atx1 mutant with overexpression of ATX1-CG (atx1-CG) or N-terminal truncated ATX1 (atx1-no N) under excess Cu stress (A) or Cu deficient conditions (B). Plants were grown on one-half-strength MS agar plates and treated with 35 μM CuSO₄ (A) or without Cu and in the presence of 10 or 50 μM Cu chelator BSA for 17 d (B).

FIG. 16 shows the sequence alignment of Cu chaperones (named ATX1 or CCH) among various plant species and yeast, having about 30% to 90% identity, wherein the Cu chaperones from Arabidopsis, Hevea, Jatropha, Polulus, Zea, and Oryza, share a higher identity (more than 80%).

### DETAILED DESCRIPTION OF THE INVENTION

Unless defined otherwise, all technical and scientific terms used herein have the same meanings as is commonly understood by one of skill in the art to which this invention belongs.

As used herein, the articles “a” and “an” refer to one or more than one (i.e., at least one) of the grammatical object of the article. By way of example, “an element” means one element or more than one element.

The term “polynucleotide” or “nucleic acid” refers to a polymer composed of nucleotide units. Polynucleotides include naturally occurring nucleic acids, such as deoxyribonucleic acid (“DNA”) and ribonucleic acid (“RNA”) as well as nucleic acid analogs including those which have non-naturally occurring nucleotides. Polynucleotides can be synthesized, for example, using an automated DNA synthesizer. The term “nucleic acid” typically refers to large polynucleotides. It will be understood that when a nucleotide sequence is represented by a DNA sequence (i.e., A, T, G, C), this also includes an RNA sequence (i.e., A, U, G, C) in which “U” replaces “T.” The term “cDNA” refers to a DNA that is complementary or identical to an mRNA, in either single stranded or double stranded form.

The term “complementary” refers to the topological compatibility or matching together of interacting surfaces of two polynucleotides. Thus, the two molecules can be described as complementary, and furthermore the contact surface characteristics are complementary to each other. A first polynucleotide is complementary to a second polynucleotide if the nucleotide sequence of the first polynucleotide is identical to the nucleotide sequence of the polynucleotide binding partner of the second polynucleotide. Thus, the polynucleotide whose sequence 5′-TATAC-3′ is complementary to a polynucleotide whose sequence is 5′-GTATA-3′.”

The term “encoding” refers to the inherent property of specific sequences of nucleotides in a polynucleotide (e.g., a gene, a cDNA, or an mRNA) to serve as templates for synthesis of other polymers and macromolecules in biological processes having either a defined sequence of nucleotides (i.e., rRNA, tRNA and mRNA) or a defined sequence of amino acids and the biological properties resulting therefrom. Therefore, a gene encodes a protein if transcription and translation of mRNA produced by that gene produces the protein in a cell or other biological system. It is understood by a skilled person that numerous different polynucleotides and nucleic acids can encode the same polypeptide as a result of the degeneracy of the genetic code. It is also understood that skilled persons may, using routine techniques, make nucleotide substitutions that do not affect the polypeptide sequence encoded by the polynucleotides described there to reflect the codon usage of any particular host organism in which the polypeptides are to be expressed. Therefore, unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “recombinant polypeptide” refers to a polynucleotide or nucleic acid having sequences that are not naturally joined together. A recombinant nucleic acid may be present in the form of a vector. “Vectors” may contain a given nucleotide sequence of interest and a regulatory sequence. Vectors may be used for expressing the given nucleotide sequence or maintaining the given nucleotide sequence for replicating it, manipulating it or transferring it between different locations (e.g., between different organisms). Vectors can be introduced into a suitable host cell for the above mentioned purposes.

As used herein, the term “operably linked” may mean that a polynucleotide is linked to an expression control sequence in such a manner to enable expression of the polynucleotide when a proper molecule (such as a transcriptional factor) is bound to the expression control sequence.

As used herein, the term “expression control sequence” or “regulatory sequence” means a DNA sequence that regulates the expression of the operably linked nucleic acid sequence in a certain host cell.

Examples of vectors include, but are not limited to, plasmids, cosmids, phages, YACs or PACs. Typically, in vectors, the given nucleotide sequence is operatively linked to the regulatory sequence such that when the vectors are introduced into a host cell, the given nucleotide sequence can be expressed in the host cell under the control of the regulatory sequence. The regulatory sequence may comprises, for example and without limitation, a promoter sequence (e.g., the cytomegalovirus (CMV) promoter, simian virus 40 (SV40) early promoter, T7 promoter, and alcohol oxidase gene (AOX1) promoter), a start codon, a replication origin, enhancers, an operator sequence, a secretion signal sequence (e.g., α-mating factor signal) and other control sequence (e.g., Shine-Dalgano sequences and termination sequences). Preferably, vectors may further contain a marker sequence (e.g., an antibiotic resistant marker sequence) for the subsequent screening procedure. For purpose of protein production, in vectors, the given nucleotide sequence of interest may be connected to another nucleotide sequence other than the above-mentioned regulatory sequence such that a fused polypeptide is produced and beneficial to the subsequent purification procedure. Said fused polypeptide includes, but is not limited to, a His-tag fused polypeptide and a GST fused polypeptide.

Where the expression vector is constructed for a plant cell, several suitable promoters known in the art may be used, including but not limited to the Figwort mosaic virus 35S promoter, the cauliflower mosaic virus (CaMV) 35S promoter, the commelina yellow mottle virus promoter, the rice cytosolic triosephosphate isomerase (TPI) promoter, the rice actin 1 (Act1) gene promoter, the uniquitin (Ubi) promoter, the rice amylase gene promoter, the adenine phosphoribosyltransferase (APRT) promoter of Arabidopsis, the mannopine synthase and octopine synthase promoters.

To prepare a transgenic plant, it is preferably that the expression vector as used herein carries one or more selection markers for selection of the transformed plants, for example, genes conferring the resistance to antibiotics such as hygromycin, ampicillin, gentamycine, chloramphenicol, streptomycin, kanamycin, neomycin, geneticin and tetracycline, URA3 gene, genes conferring the resistance to any other toxic compound such as certain metal ions or herbicide, such as glufosinate or bialaphos.

As used herein, the term “transgenic plant” or “transgenic line” refers to a plant that contains a recombinant nucleotide sequence. The transgenic plant can be grown from a recombinant cell.

A variety of procedures that can be used to engineer a stable transgenic plant are available in this art. In one embodiment of the present invention, the transgenic plant is produced by transforming a tissue of a plant, such as a protoplast or leaf-disc of the plant, with a recombinant Agrobacterium cell comprising a polynucleotide encoding an ATX1-like protein as described herein and generating a whole plant from the transformed plant tissue. In another embodiment, a polynucleotide encoding a desired protein can be introduced into a plant via gene gun technology, particularly if transformation with a recombinant Agrobacterium cell is not efficient in the plant.

The term “polypeptide” refers to a polymer composed of amino acid residues linked via peptide bonds. The term “protein” typically refers to relatively large polypeptides. The term “peptide” typically refers to relatively short polypeptides.

It is understandable a polypeptide may have a limited number of changes or modifications that may be made within a certain portion of the polypeptide irrelevant to its activity or function and still result in a molecule with an acceptable level of equivalent biological activity or function. Modifications and changes may be made in the structure of such polypeptides and still obtain a molecule having similar or desirable characteristics. For example, certain amino acids may be substituted for other amino acids in the peptide/polypeptide structure (other than the conserved region) without appreciable loss of activity. Amino acid substitutions are generally based on the relative similarity of the amino acid side-chain substituents, for example, their hydrophobicity, hydrophilicity, charge, size, and the like. For example, arginine (Arg), lysine (Lys), and histidine (His) are all positively charged residues; and alanine (Ala), glycine (Gly) and serine (Ser) are all in a similar size. Therefore, based upon these considerations, arginine (Arg), lysine (Lys) and histidine (His); and alanine (Ala), glycine (Gly) and serine (Ser) may be defined as biologically functional equivalents. One can readily design and prepare recombinant genes for microbial expression of polypeptides having equivalent amino acid residues.

To determine the percent identity of two amino acid sequences, the sequences are aligned for optimal comparison purposes (e.g., gaps can be introduced in the sequence of a first amino acid sequence for optimal alignment with a second amino acid sequence). In calculating percent identity, typically exact matches are counted. The determination of percent homology or identity between two sequences can be accomplished using a mathematical algorithm known in the art, such as BLAST and Gapped BLAST programs, the NBLAST and XBLAST programs, or the ALIGN program.

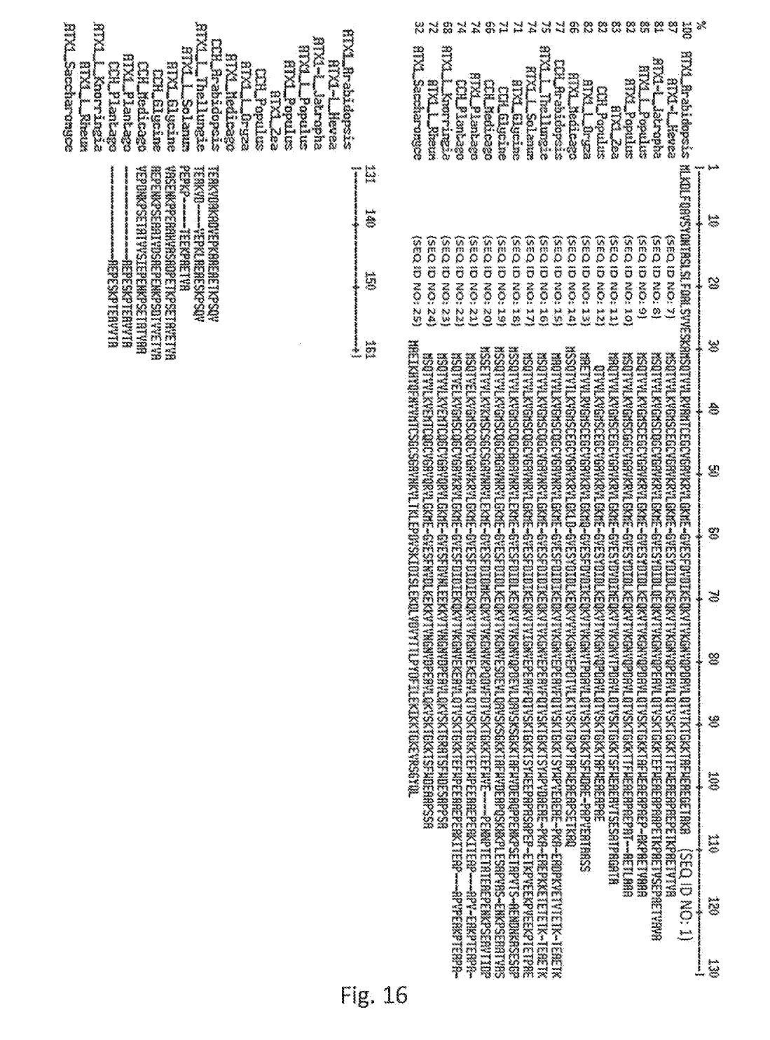

“ATX1 protein” is known as a Cu chaperone that has been found in many species of organisms (e.g. yeast, plant or bacteria), having a conserved MTCXXC motif (X, any residue) for Cu binding activity (Pufahl et al., 1997; Shoshan and Tshuva, 2011). In this art, the Cu chaperones from different species are known to be named ATX1 or CCH. As shown in FIG. 16, Cu chaperones (named ATX1 or CCH) among various plant species and yeast, have about 30% to 90% identity, with the conserved MTCXXC motif, wherein the Cu chaperones from Hevea, Jatropha, Polulus, Zea, and Oryza, have a higher identity (more than 80%), when compared to ATX1 from Arabidopsis (SEQ ID NO: 1). The Cu binding activity of ATX1 protein can be assayed using various methods known in the art in view of the present disclosure, such as a yeast two-hybrid experiment.

In this invention, it is demonstrated that transgenic plants overexpressing Arabidopsis ATX1 (AtATX1, SEQ ID NO: 1) exhibit tolerance to excess Cu and Cu deficiency, wherein not only the Cu-binding motif MXCXXC (residues 41-46) but also the N-terminal sequence (residues 1-30) are required for such physiological functions. According to the invention, “ATX1-like polypeptide” as used herein refers to a polypeptide which has an amino acid sequence with at least 30% identity to AtATX1 (SEQ ID NO: 1) and exhibits a copper binding activity, in which the amino acid sequence of the ATX1-like polypeptide comprises (i) a N-terminal sequence that is identical to SEQ ID NO: 2 or has an amino acid sequence greater than 60% identity to SEQ ID NO: 2, and (ii) a C-terminal sequence that comprises the Cu-binding motif MXCXXC (SEQ ID NO: 3).

Accordingly, in one aspect, the present invention provides a transgenic plant transformed with a recombinant polynucleotide comprising a nucleotide sequence encoding an ATX1-like polypeptide, operatively linked to an expression control sequence, wherein the ATX1-like polypeptide has an amino acid sequence having at least 30%, 35%, 40%, 45%, 50%, 55%, 60%, 65%, 70%, 75%, 80%, 85%, 90%, or 95% identity to SEQ ID NO: 1 and exhibits a copper binding activity, in which the amino acid sequence of the ATX1-like polypeptide has (i) an N-terminal sequence having at least at least 60%, 65%, 70%, 75%, 80%, 85%, 90%, or 95% identity to SEQ ID NO: 2, corresponding to positions 1 to 30 of SEQ ID NO: 1, and (ii) a C-terminal sequence comprising a copper binding motif of SEQ ID NO: 3 (MXCXXC), corresponding to positions 41 to 46 of SEQ ID NO: 1.

In some embodiments, the N-terminal sequence is SEQ ID NO: 2.

In some embodiments, the C-terminal sequence comprises SEQ ID NO: 4, corresponding to positions 31 to 106 of SEQ ID NO: 1.

In some embodiments, the C-terminal sequence is selected from the group consisting of SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23 and SEQ ID NO: 24.

In other embodiments, the C-terminal sequence comprises SEQ ID NO: 5. In one example, the C-terminal sequence is SEQ ID NO: 25.

In particular embodiments, the ATX1-like polypeptide has 90 or more (e.g. 90 to 250, or 90 to 225, or 90 to 200, or 90 to 190, or 90 to 180, or 90 to 170) consecutive amino acid residues in total length, in which the N-terminal sequence covers residues at positions 1-30 and the C-terminal sequence, fused with the N-terminal sequence, covers residues from position 31 to the end.

In some embodiments, the ATX1-like polypeptide is composed of SEQ ID NO: 2 as the N-terminal sequence, fused with a C-terminal sequence selected from the group consisting of SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24 and SEQ ID NO: 25.

In one particular embodiment, the ATX1-like protein has the amino acid sequence of SEQ ID NO: 1.

In one particular embodiment, the nucleotide sequence encoding the ATX1-like protein is SEQ ID NO: 26.

Plants to which the present invention can be applied include both monocotyledon and dicotyledon. Examples of monocotyledon includes but not limited to rice, barley, wheat, rye, oat, corn, bamboo, sugar cane, onion, leek and ginger. Examples of the dicotyledons include, but are not limited to Arabidopsis thaliana, eggplant, tobacco plant, red pepper, tomato, burdock, crown daisy, lettuce, balloon flower, spinach, chard, sweet potato, celery, carrot, water dropwort, parsley, Chinese cabbage, cabbage, radish, watermelon, melon, cucumber, pumpkin, gourd, strawberry, soybean, mung bean, kidney bean, and pea.

According to the invention, a transgenic plant overexpressing an ATX-1 like polypeptide is resistant to excess Cu or Cu deficiency and can accumulate Cu in a higher level, as compared with a wild type plant (non-transgenic) while being grown under the same conditions. As used herein, excess Cu can mean its concentration is higher than a regular amount, by about 150%, 200%, 250%, 300%, 400% or more, for plant growth. Cu deficiency can mean its concentration is lower than a regular amount, e.g., 80%, 70%, 60%, 50%, 40%, 30%, 20%, or 10% of a regular amount or less, for plant growth. For example, an amount of 6 ppm Cu can be understood as an adequate concentration, and ≧20 ppm can induce toxicity in shoot tissues (see for example Marschner, 1995; Burkhead et al., 2009).

In some embodiments, the transgenic plant overexpressing an ATX-1 like polypeptide can accumulate Cu in a level higher by about 110% or higher, by about 120% or higher, by about 140% or higher, by about 160% or higher, by about 180% or higher, by about 200% or higher, than a wild type plant (non-transgenic) while being grown under the same conditions. The transgenic plant also exhibits enhanced growth

Thus, the present invention also provides a method for producing a transgenic plant, comprising (a) transforming a plant cell with a recombinant polynucleotide comprising a nucleotide sequence encoding an antioxidant protein 1 (ATX1)-like polypeptide, as described herein, to obtain a recombinant plant cell; and (b) growing the recombinant plant cell obtained in (a) to generate a transgenic plant.

To select a pant with desired traits, the method of the invention further comprises (c) selecting a transgenic plant which is resistant to excess Cu or Cu deficiency or can accumulate Cu in a higher level, as compared with a wild type plant (non-transgenic) while being grown under the same conditions.

In a further aspect, the present invention provides a method of phytoremediation by using the transgenic plant of the invention as described herein to remove Cu contamination.

In particular, the method of phytoremediation of the invention comprises:

(a) selecting an environment contaminated with copper; and

(b) growing, in said environment, a transgenic plant transformed with a recombinant polynucleotide comprising a nucleotide sequence encoding an antioxidant protein 1 (ATX1)-like polypeptide, operatively linked to an expression control sequence, wherein the ATX-like polypeptide has an amino acid sequence having at least 30%, 35%, 40%, 45%, 50%, 55%, 60%, 65%, 70%, 75%, 80%, 85%, 90%, or 95% identity to SEQ ID NO: 1 and exhibits a copper binding activity, in which the amino acid sequence of the ATX-like polypeptide has (i) an N-terminal sequence having at least 60%, 65%, 70%, 75%, 80%, 85%, 90%, or 95% identity to SEQ ID NO: 2, corresponding to positions 1 to 30 of SEQ ID NO: 1, and (ii) a C-terminal sequence comprising a copper binding motif of SEQ ID NO: 3, corresponding to positions 41 to 46 of SEQ ID NO: 1, wherein the transgenic plant accumulates and removes an amount of copper from the environment.

Also provided is a method for enhancing growth of a plant by introducing to a plant cell a recombinant polynucleotide comprising a nucleotide sequence encoding the ATX1-like polypeptide as described herein to produce a transgenic plant and growing such transgenic plant. Especially, the transgenic plant can grow in a copper deficient condition, without additional supply of copper.

Accordingly, the method of the invention provides a variety of advantages at least including (i) it is environmentally friendly, coat-effective, and aesthetically pleasing (ii) the metals absorbed by the plants may be extracted from harvested plant biomass and then sustainably recycled, (iii) phytoremediation can be used to clean up a large variety of contaminants; (iv) the entry of contaminants into the environment is reduced by preventing their leakage into the groundwater systems; and (v) biomass is increased in nutrient insufficient conditions.

The present invention is further illustrated by the following examples, which are provided for the purpose of demonstration rather than limitation. Those of skill in the art should, in light of the present disclosure, appreciate that many changes can be made in the specific embodiments which are disclosed and still obtain a like or similar result without departing from the spirit and scope of the invention.

1. Materials and Methods

1.1 Plant Growth Conditions

The procedure was modified from a previous study (Chen et al., 2011). Seeds of wild-type Arabidopsis thaliana (ecotype Columbia-0), cch T-DNA insertion line (SALK_{—}138593), atx1 T-DNA insertion line (SALK_{—}026221), from the Arabidopsis Biological Resource Center, and cchatx1 double cross from SALK_{—}138593 and SALK_{—}026221 were surface-sterilized with 70% ethanol for 5 min, then treated with 1.2% bleach containing 0.02% SDS for 15 min, rinsed 5 times with sterilized water, and kept in darkness at 4° C. for 3 d for seed stratification. Sterilized seeds were grown on half-strength MS medium (½ MS salt; Sigma-Aldrich, St. Louis, Mo.), 1% sucrose (J. T. Baker, Phillipsburg, N.J.), 0.5 g/L of 2-morpholinoethanesulfonic acid (MES, J. T. Baker) and 0.7% agar (Sigma-Aldrich, A.7002) at pH 5.7 for designated times. Chemical treatment is described in figure legends. Seeds were grown (after 3 d of stratification) in pots containing organic substrate, vermiculite and mica shoot at a ratio of 9:1:1 at a light intensity of 100 μmol m⁻² s⁻¹ under a 16-h light/8-h dark cycle at 22° C.

1.2 Overexpression of CCH and ATX1

Agrobacterium tumefaciens, strain GV3101, harboring the plasmids 35S:AtCCH/pCAMBIA1305.1 or 35S:AtATX1/pCAMBIA1305.1 to overexpress the coding sequence (CDS) including CCH or ATX1 of Arabidopsis driven by a CaMV35S promoter was transformed into plants with a cch or atx1 mutant background. For overexpressing CCH or ATX1 in the wild type, the same constructs were transformed into a wild-type background. For PCR amplification of CDS, the following primer pairs were used:

TABLE 1
Primers for PCR amplification of CDS

FP-CCH-NcoI
SEQ ID NO: 27
5′-AAC CAT GGG GAT GGC TCA GAC CGT TGT CCT
CA-3′
RP-CCH-Pm1I
SEQ ID NO: 28
5′-AAC ACG TGT TAA ACT TGT GAT GGC TTA GTC
T-3′
FP-ATX1-NcoI
SEQ ID NO: 29
5′-AAC CAT GGA TGC TTA AAG ACT TGT TCC
AAG-3′
RP-ATX1 -Pm1I
SEQ ID NO: 30
5′-AAC ACG TGT TAA GCC TTA GCA GTT TCA CCT
TC-3′

1.3 Protein Extraction and Immunoblotting Analysis of CCH and ATX1

The procedure was described previously (Chen et al., 2011). Plant samples were extracted with the extraction buffer (2×SDS sample buffer containing 20 mM N-ethylmaleimide, 100 mM Na₂S₂O₅ and one tablet of protease inhibitor cocktail [Roche Applied Science, Mannheim, Germany] per 50 ml). Samples were centrifuged at 12,000×g for 10 min, and the protein concentration was determined by use of the BCA Protein Assay Kit (Thermo Scientific). Total protein (10 μg) was separated on a NuPAGE 4-12% Bis-Tris Gel (Invitrogen) and transferred to a PVDF membrane (Immobilon-P, Millipore), which was blocked with 5% fat-free milk and 0.1% Tween 20 in PBS for 1 h, incubated with 1:5,000-diluted purified anti-CCH or -ATX1 antibody, washed with PBS buffer containing 0.1% Tween 20 (PBST), and incubated for 1 h with 1:10,000-diluted secondary antibody (peroxidase-conjugated goat anti-rabbit IgG; Millipore Corp., Temecula, Calif., USA). The membrane was washed 5 times for 10 min each with PBST solution before development. Specific protein bands were visualized by use of the Immobilon Western Chemiluminescent HRP substrate (Millipore Corp., Billerica, Mass., USA).

1.4 Elemental Analysis

Elemental analysis was as described (Lin et al., 2009). Harvested plant samples were washed with CaCl₂ and H₂O and dried for 3 days before digestion. Microwave-digested samples (CEM, Matthews, N.C., USA) were analyzed by inductively coupled plasma-optical emission spectrometry (ICP-OES) (OPTIMA 5300; Perkin-Elmer, Wellesley, Mass., USA).

1.5 RNA Isolation and Quantitative Real-Time RT-PCR (qPCR)

The procedure was described previously (Chen et al., 2011). Frozen root tissues were ground in liquid nitrogen by use of a tissue homogenizer (SH-48, J&H Technology Co.). Total RNA was isolated by the TRIzol method. RNA was precipitated by adding 0.5 mL isopropanol and incubating at −80° C. for 30 min Following centrifugation at 15,000×g at 4° C. for 15 min, the resulting pellet was washed twice with 75% ethanol. RNA was redissolved in 30 μl of DEPC-treated H₂O. The concentration of the RNA was determined at 260 nm on a NanoDrop ND-1000 Spectrophotometer (Isogen Life Science, De Meern, The Netherlands). Subsequently, 2 μg RNA was treated with RQ1 RNase-Free DNase (Promega), and the reaction buffer was replaced with 5× First-strand RT Buffer (Invitrogen). The cDNA was synthesized by use of SuperScript III Reverse Transcriptase (Invitrogen). qPCR analyses involved use of SYBR Green I Dye (ABI). The expression of Actin2 (ACT2) was used as the internal control for all tested genes. The sequences of primers are in Table 2.

TABLE 2
Primers used for qPCR in determining HMA5 and COPT1
expression

| Target | Primer sequence | Position | Length (bp) | Tm (° C.) | GC % |
|---|---|---|---|---|---|
| AtHMA5 | F: 5′-TGGCCAGAAGCCTGTGATTT-3′ | 2164-2184 | 20 | 59 | 50 |
| | (SEQ ID NO: 31) | | | | |
| | R: 5′-TGGCTTTCACTCCCTTTCC-3′ | 2215-2235 | 20 | 59 | 50 |
| | (SEQ ID NO: 32) | | | | |
| AtCOPT1 | F: 5′-GCCGTTGGTTTCATGTTGTTC-3′ | 472-493 | 21 | 59 | 48 |
| | (SEQ ID NO: 33) | | | | |
| | R: 5′-TTTTCCGGTCATGGAGGT-3′ | 532-551 | 19 | 59 | 53 |
| | (SEQ ID NO: 34) | | | | |
| AtATC2 | F: 5′-AGGTCCAGGAATCGTTCACAGA-3′ | 1407-1429 | 22 | 60 | 50 |
| | (SEQ ID NO: 35) | | | | |
| | R: 5′-CCCCAGCTTTTAAGCCTTTGA-3′ | 1452-1474 | 22 | 60 | 46 |
| | (SEQ ID NO: 36) | | | | |

1.6 Photosynthetic Activity Assay

The maximum quantum yield (Fv/Fm) was measured by use of a portable chlorophyll florometer PAM-2100 (Heinz Walz, Germany).

1.7 MDA Content Quantification

An amount of 0.05 g shoot or root tissue was homogenized with 2 mL of 0.1% (w/v) cool trichloroacetic acid (TCA) on ice. The homogenates were centrifuged at 14,000×g for 10 min at 4° C., then 250 μL supernatant was mixed with 1.5 mL TCA/TBA reagent (0.25% TBA containing 10% TCA). The mixture was incubated in a water heater at 95° C. for 30 min, kept on ice for 5 min, then centrifuged at 3000×g for 10 min; then 200 μL of supernatant containing MDA equivalents was monitored by measuring the absorbance at 532, 600 and 440 nm by spectrophotometry (BioTek, Winooski, Vt., USA). MDA content was calculated as follows: (A₅₂₃-A₅₆₀)/155 (K mM⁻¹ cm⁻¹)*5*4*1000/FW (g).

1.8 Peroxidase and Catalase Activity Assay

Shoot or root tissue was homogenized with liquid nitrogen and suspended in 0.1 mL of 10 mM PBS buffer (pH 7.0). The homogenates were centrifuged at 14,000×g for 20 min, and the supernatant was collected for analysis. Peroxidase (PDX) activity was determined by measuring the increase in absorbance at 470 nm after 20-min incubation at room temperature by spectrophotometry (BioTek). The reaction mixture was 25 μL of 50 mM H₂O₂, 5 μL of 250 mM guaiacol, 195 μL of 12.5 mM 3, 3-dimethylglutaric acid (pH 6.0) and 25 μL protein extracts. The reaction was started by adding 100 μl protein extract to 900 μl reaction solution. One unit of PDX isoenzymes was defined as the amount of enzyme that could produce 1 nmol tetraguaiacol per min (extinction coefficient is 26.6 mM⁻¹cm⁻¹ at 470 nm). Catalase (CAT) activity was determined by monitoring the decrease in absorbance at 240 nm at room temperature by spectrophotometry. The reaction mixtures contained 5 mM H₂O₂ in 50 mM PBS buffer (pH 7.0). The reaction was started by adding 100 μl protein extract to 900 μl reaction solution. One unit of CAT was defined as the amount of enzyme able to decompose 1 μmol of H₂O₂ in 1 min at 25° C. (extinction coefficient is 0.039 mM⁻¹ cm⁻¹ at 240 nm).

1.9 Statistical Analysis

Student t test was used for statistical analysis. P<0.05 was considered statistically significant.

2. Results

2.1 Isolation of Cu Chaperone Mutants

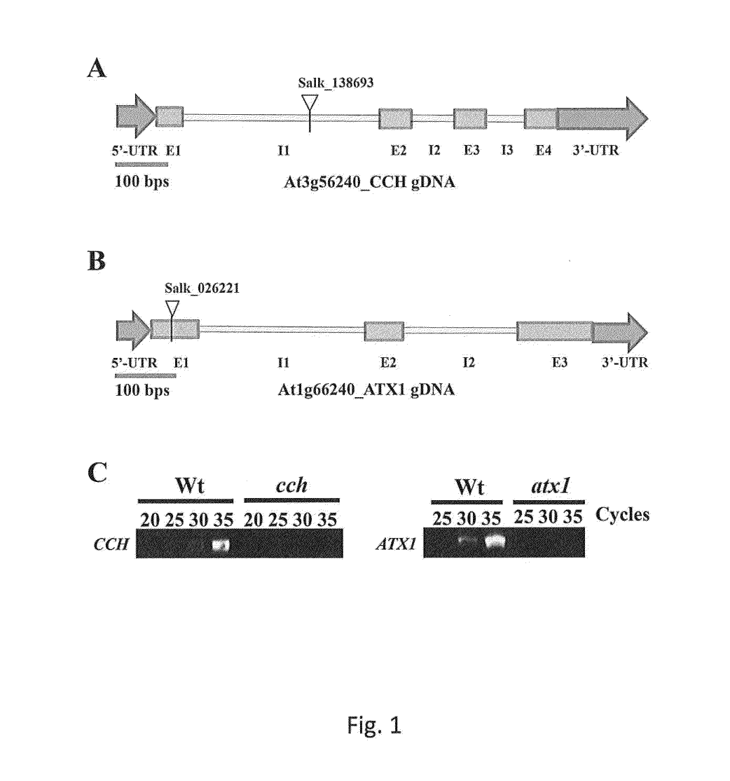

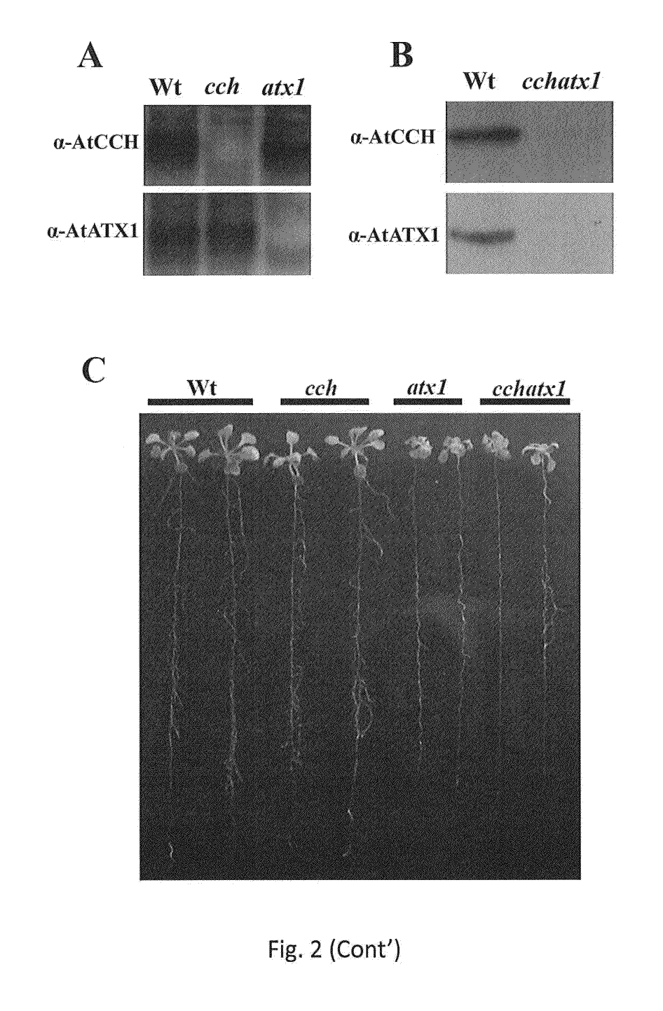

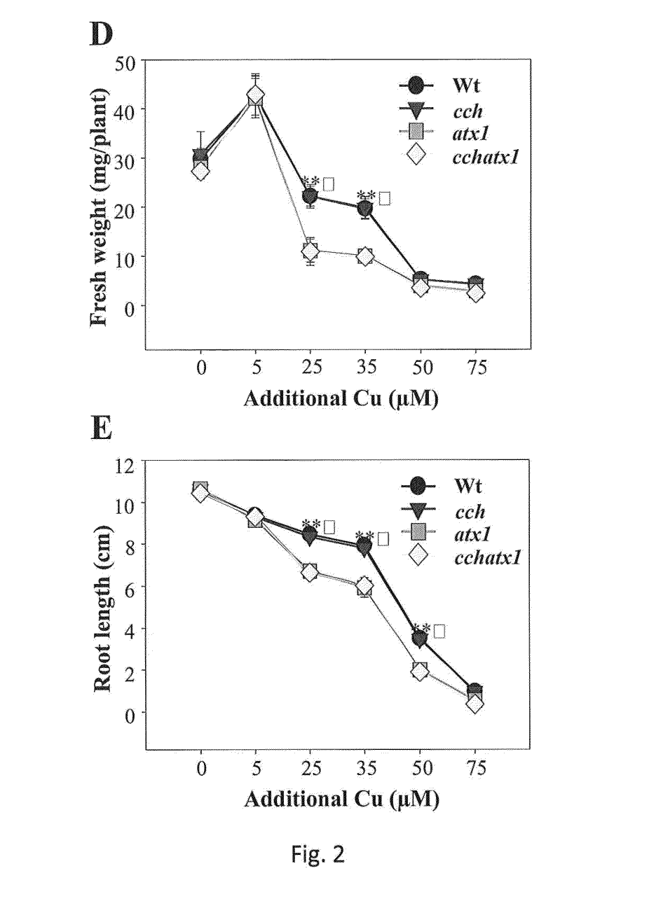

To examine the biological function of CCH and ATX1, we used Arabidopsis mutants with T-DNAs inserted in CCH (Salk_{—}138593) and AIX1 (Salk_{—}026221) (FIGS. 1A and 1B). RT-PCR used to analyze the expression of CCH and ATX1 revealed no signals in cch or atx1 mutants (FIG. 1C), so the T-DNA insertions resulted in complete loss of gene expression in these mutants. To confirm the null function of both genes, we generated antibodies against CCH and ATX1 and found neither CCH nor ATX1 accumulated in the cch or atx1 mutants, respectively (FIG. 2A). The cchatx1 double mutant, created by crossing the cch and atx1 mutants, showed no CCH or ATX1 protein accumulation (FIG. 2B). We used these Cu chaperone mutants for phenotypic characterization.

2.2 The atx1 and cchatx1 Mutants are Highly Sensitive to Excess Cu

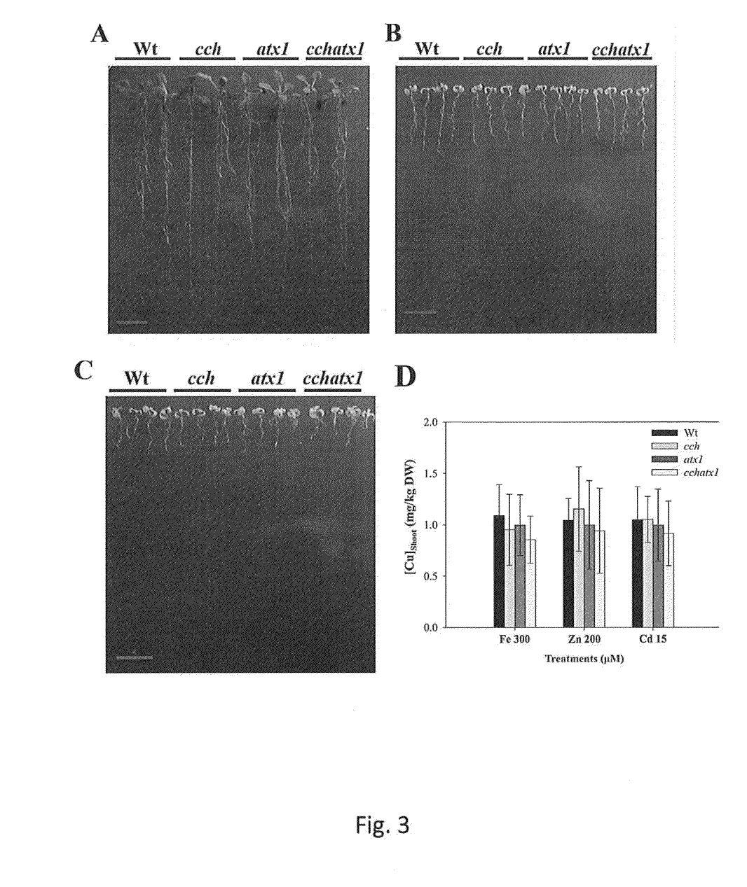

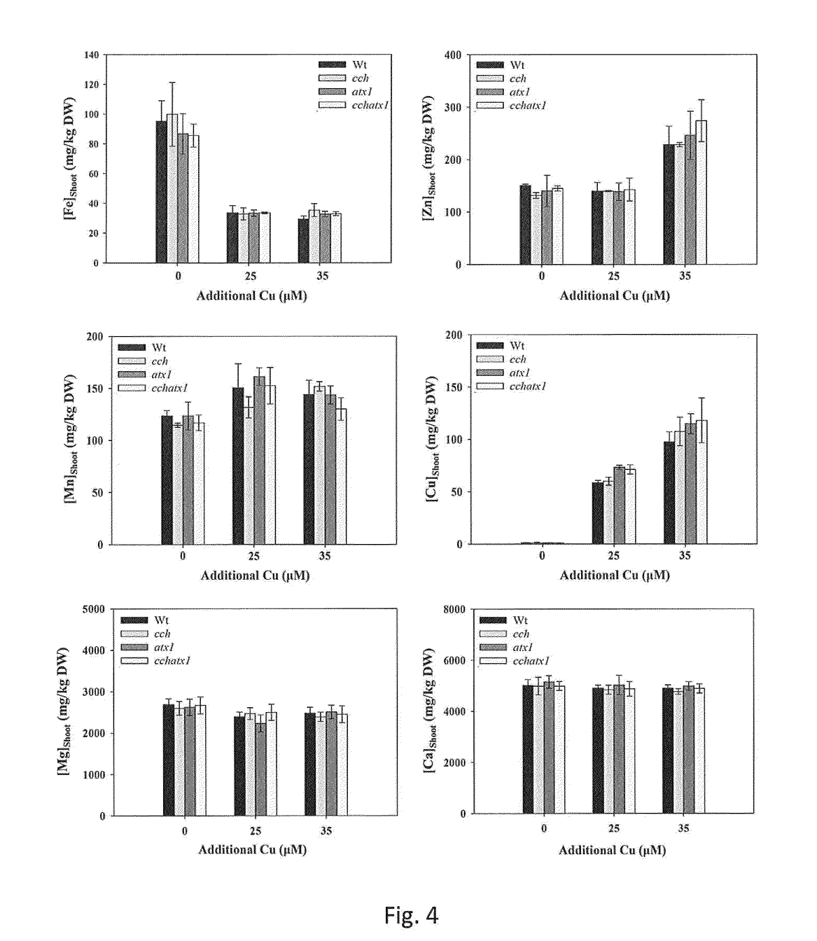

To study the biological role of CCH and AIX1 in plant development, we analyzed tolerance to Cu, Fe, Zn, and Cd stresses; triple responses to ethylene treatment; and responses to paraquat, heat and cold shock in the wild type and Cu chaperone mutants (Lin and Culotta, 1995; Woeste and Kieber, 2000; Shibasaki et al., 2009; Liu et al., 2011) in terms of plant biomass and root length (Marschner, 1995; Lequeux et al., 2010). The atx1 and cchatx1 mutants were hypersensitive to excess Cu among the heavy metals in root length and growth (FIG. 2 and FIG. 3). The other treatments produced no obvious phenotype (data not shown). Fresh weight and root length were lower for atx1 and cchatx1 than the wild type and cch mutant with excess Cu (FIGS. 2D and 2E). The degrees of growth reduction for both atx1 and cchatx1 were almost identical, which suggests no added effects with the cch defect. With 25 and 35 μM Cu, the fresh weight for both atx1 and cchatx1 was 49% and 51%, respectively, that of the wild type. As well, with 25, 35 and 50 μM Cu, the root length was about 80%, 76% and 57%, respectively, that of the wild type. Of note, shoot Cu accumulation was similar in wild type and mutants grown in half-strength MS media with excess Cu or other heavy metals (FIG. 3). As well, the wild type and mutants did not differ in shoot Fe, Zn, Mn, Mg or Ca accumulation with excess Cu (FIG. 4). In summary, atx1 and cchatx1 mutants were specifically sensitive to Cu stress under our tested conditions. The response of cch to excess Cu was similar to that of the wild type. Therefore, ATX1 but not CCH is involved in Cu tolerance in Arabidopsis.

2.3 Expression of CCH and ATX1 Is Independent of Each Other

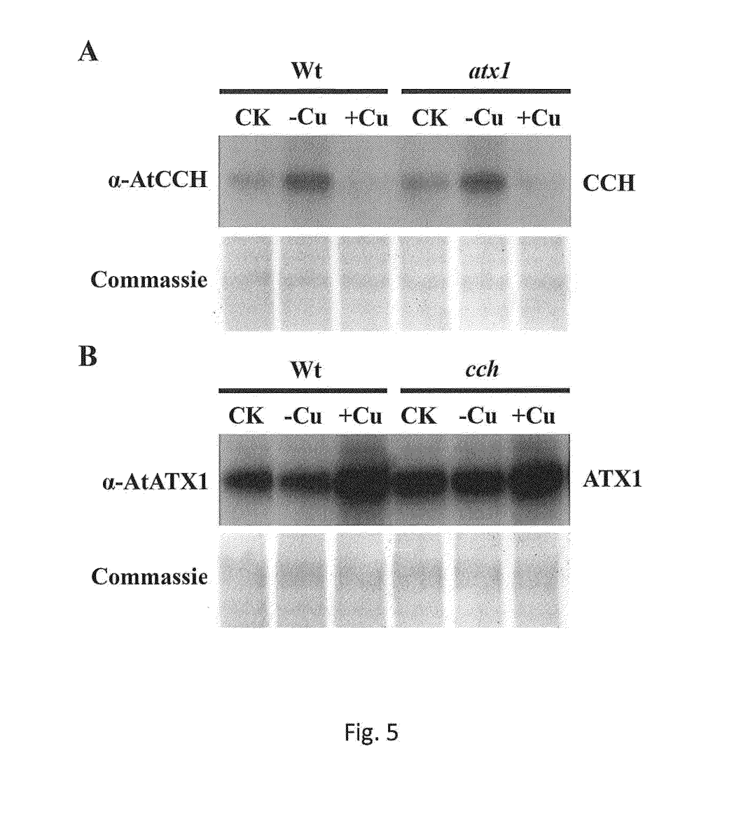

Both CCH and ATX1 are predicted to contribute to Cu homeostasis, and their expression is influenced by Cu availability (Mira et al., 2001; Puig et al., 2007). However, whether they affect each other's expression is not known. We examined the protein accumulation of ATX1 and CCH in cch and atx1 mutants, respectively, under different Cu conditions. CCH expression was induced by Cu deficiency and reduced with excess Cu, whereas ATX1 expression was induced with excess Cu (FIG. 5). These data support previous mRNA accumulation results (Himelblau et al., 1998; Puig et al., 2007). In addition, the ATX1 and CCH accumulation patterns in cch and atx1 were identical to those in the wild type (FIG. 5). Thus, the expression of CCH and ATX1 is independent in response to Cu excess or deficiency.

2.4 Excess Cu Negatively Affects Chlorophyll Content, Lipid Peroxidation and Antioxidant Enzymes in atx1 and cchatx1

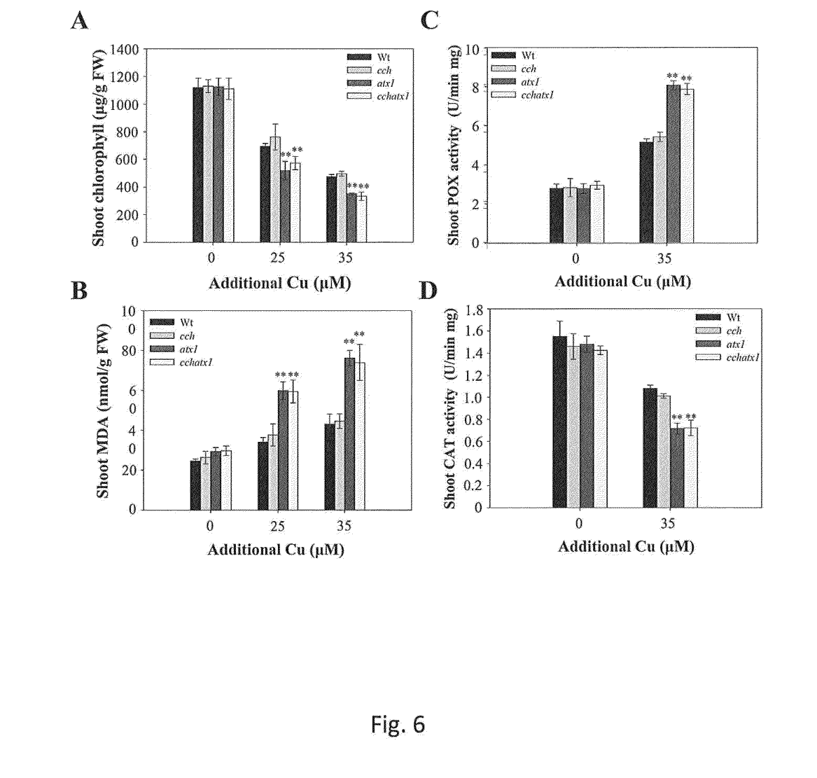

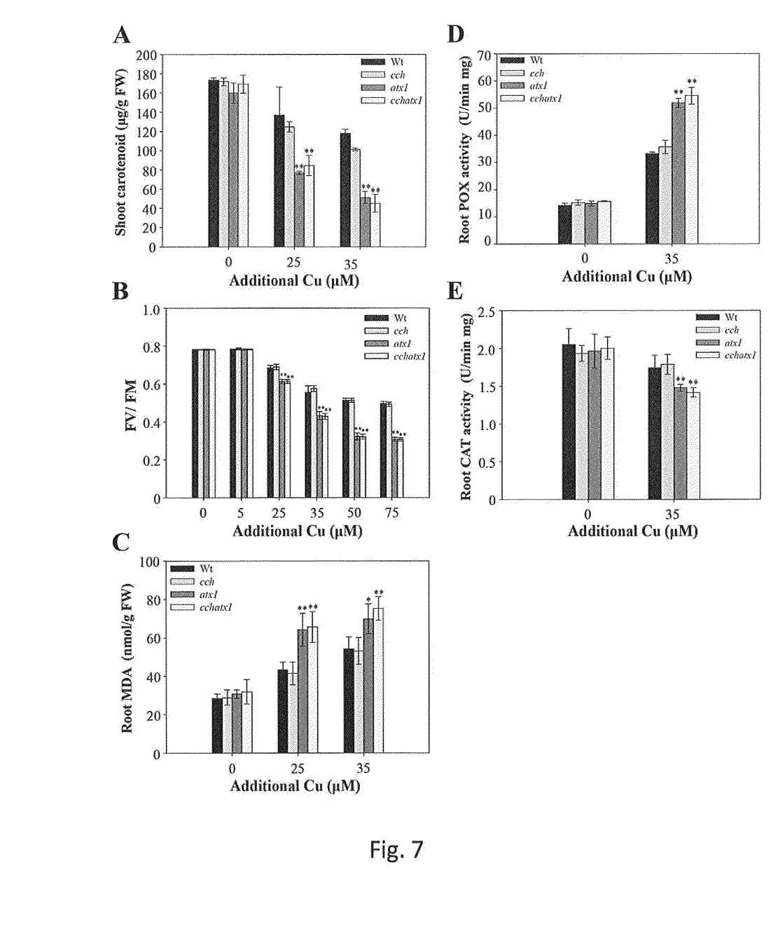

Cu toxicity initiates loss of chloroplast integrity, inhibited photosynthetic electron transport, and increased lipid peroxidation and influences antioxidant enzymes (Patsikka et al., 2002; Drazkiewicz et al., 2004; Sun et al., 2010). The most common symptom to judge loss of chloroplast integrity is chlorosis, which results from reduced chlorophyll and carotenoid contents in vegetative tissue. With leaf chlorosis in seedlings with excess Cu for 3 day, total chlorophyll content in atx1 and cchatx1 mutants was 73% of the wild-type content (FIG. 6A). Furthermore, carotenoid content was similarly reduced with excess Cu (FIG. 7A).

Photosystem II (PSII) is a primary target for Cu toxicity (Kupper et al., 2003). With excess Cu, low-efficient PSII exhibits photooxidative damage, which results in inhibited electron transport chain. We used the potential quantum yield of PSII (Fv/Fm) as an indicator of photooxidative damage. With excess Cu, the Fv/Fm ratio was significantly lower for atx1 and cchatx1 than the wild type and cch mutant (FIG. 7B). Therefore, excess Cu induces high damage to plastids in atx1 and cchatx1 mutants.

As a redox-active metal, Cu can catalyze the formation of superoxide anion (O₂⁻) and result in production of H₂O₂ and HO⁻ by Fenton reaction (Schutzendubel and Polle, 2002). These excess ROS remove electrons from the lipids of cell membranes and cause lipid peroxidation, thereby damaging cells. Malondialdehyde (MDA) is one of the final products of lipid peroxidation. MDA content has been used to estimate the degree of oxidative stress in plants with excess Cu (Cho and Sohn, 2004; Skorzynska-Polit et al., 2010). We found that with excess Cu, leaf MDA content in atx1 and cchatx1 was 175% of the wild-type content (FIG. 6B). Root MDA content was also increased in the mutants (FIG. 7C). Therefore, excess Cu induces high lipid peroxidation in atx1 and cchatx1 mutants.

According to a previous study, Cu toxicity induced the activity of peroxidase (PDX) and reduced that of catalase (CAT) in Arabidopsis (Drazkiewicz et al., 2004). We further examined the activation of PDX and CAT and found a significant increase in PDX activity in shoots and roots of Arabidopsis and especially atx1 and cchatx1 with Cu treatment (FIG. 6C and FIG. 7D). With excess Cu, the activity of PDX in atx1 and cchatx1 was about 156% and 152%, respectively, of the wild-type activity in shoots and 156% and 164%, respectively, of the wild-type activity in roots (FIG. 6C and FIG. 7D). However, with excess Cu, CAT activity in mutants was 67% of the wild-type activity in shoots and about 83% of the wild-type activity in roots (FIG. 6D and FIG. 7E). Thus, atx1 and cchatx1 mutants experienced higher oxidative stress with excess Cu than the wild type and cch mutant. ATX1 may play a crucial role in Cu tolerance by suppressing the negative effects of excess Cu.

2.5 Expression of HMA5 and COPT1 in Mutants

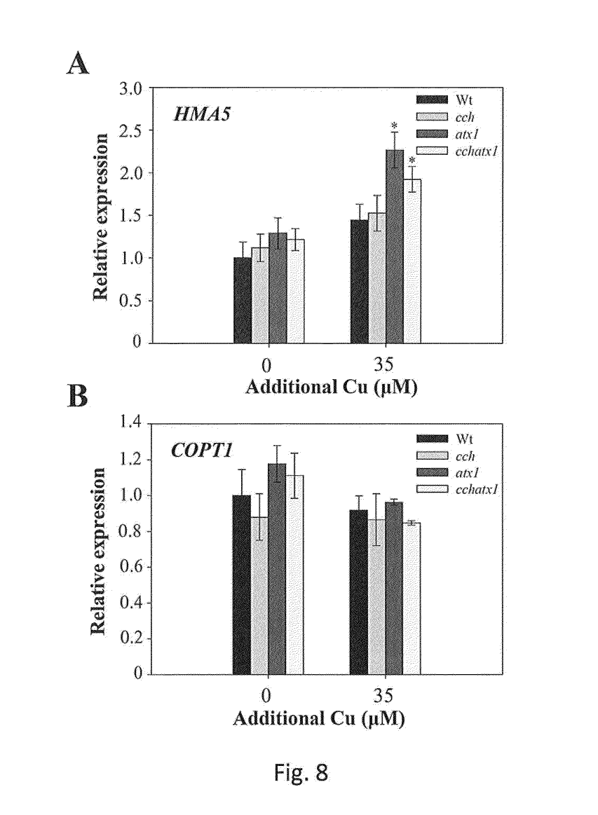

The Cu sensitive phenotype of atx1 and cchatx1 mutants was enhanced with increased Cu concentration in the medium. Increased Cu may disrupt the homeotic regulation of Cu. The balance between Cu uptake and transport mainly relies on the expression of COPT1 and HMA5 in the root, which are regulated by Cu content in Arabidopsis (Sancenon et al., 2004; Andres-Colas et al., 2006). We used quantitative RT-PCR (qPCR) to determine whether excess Cu leads to the misregulation of COPT1 and HMA5 in atx1 and cchatx1. In the 3-day-treatment, we found that excess Cu induced the HMA5 level in roots of wild type and cch about 144% and 152%, respectively (p=0.02; FIG. 8A). The induction in wild type was also observed previously in a prolong treatment (Andres-Colas et al., 2006). With excess Cu, HMA5 level was much higher in atx1 and cchatx1 than wild-type roots (FIG. 8A), but COPT1 level was similar among wild-type and mutant roots (FIG. 8B). The upregulation of HMA5 with excess Cu was thought to participate in reducing the Cu toxicity in the root (Burkhead et al., 2009). Therefore, excess Cu could induce the expression of HMA5 in atx1 and cchatx1, which confirmed that atx1 and cchatx1 mutants were adversely affected by the Cu stress.

2.6 ATX1-Overexpressed Arabidopsis Exhibits Tolerance to Excess Cu

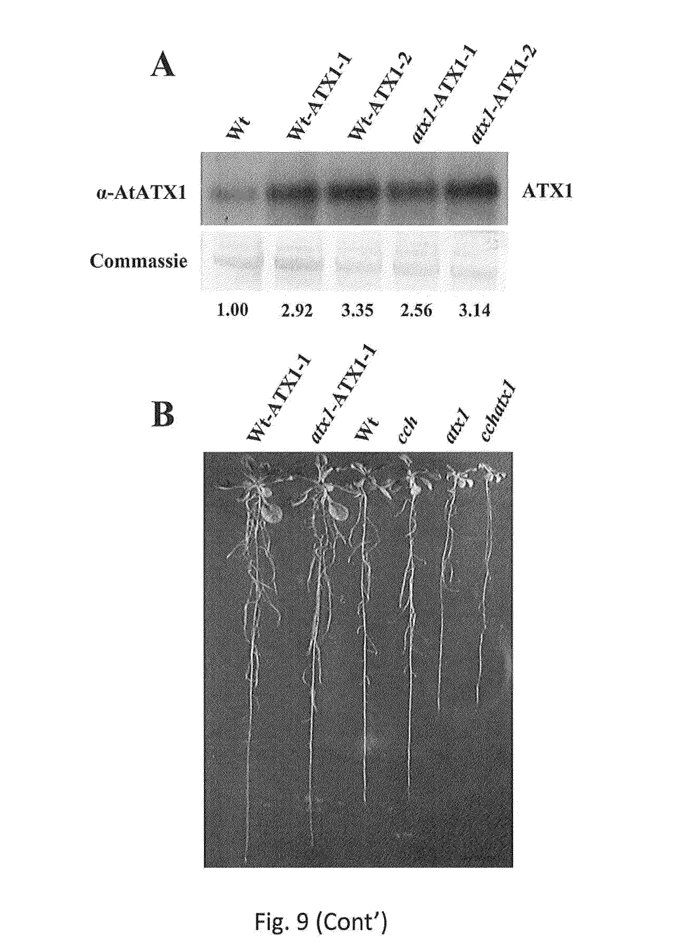

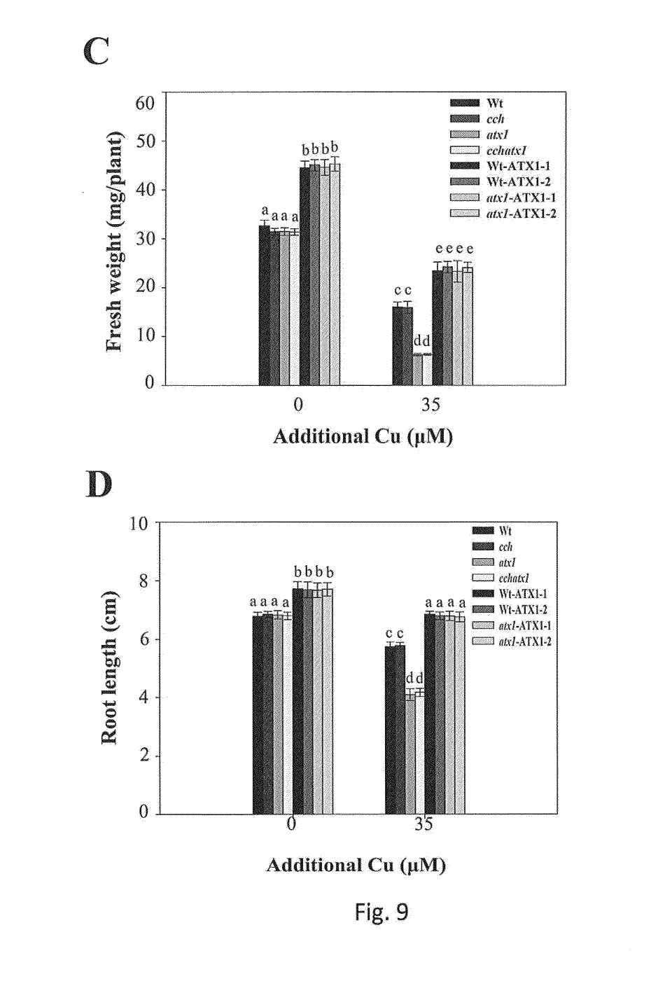

We generated Arabidopsis transgenic plants overexpressing ATX1 in a wild-type and atx1 mutant background (Wt-ATX1 and atx1-ATX1, respectively) and used immunoblotting with total proteins extracted from 14-d-old T3 homozygous plants to examine the accumulation of ATX1 protein in both Wt-ATX1 and atx1-ATX1 (FIG. 9A). To determine Cu tolerance in these transgenic lines, we measured fresh weight and root length. Overexpression of ATX1 restored the tolerance to excess Cu in the atx1 mutant (FIG. 9B). The fresh weight of transgenic plants was about 136% to 139% with half-strength MS and about 145% to 300% with excess Cu as compared with the wild type and cch (FIG. 9C). As well, with half-strength MS and excess Cu, root lengths were longer for Wt-ATX 1-1, 2 and atx1-ATX 1-1, 2 than the wild type and cch (FIG. 9D). Therefore, overexpression of ATX1 rescued the Cu-hypersensitive phenotype of atx1 and cchatx1 mutants and stimulated growth under both half-strength MS and excess Cu conditions.

2.7 ATX1-Overexpressed Arabidopsis Shows Tolerance to Cu Deficiency

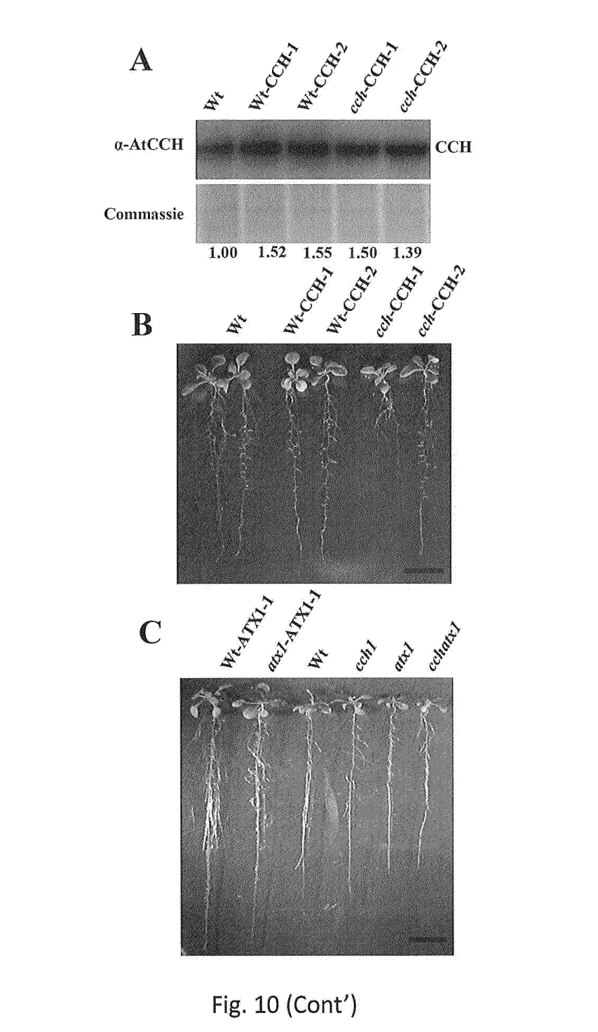

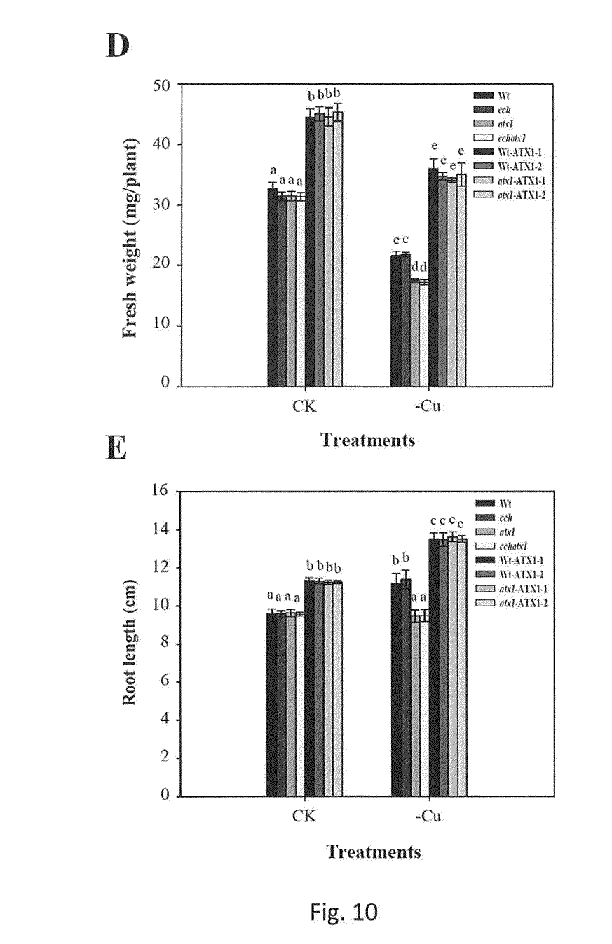

The expression of CCH was induced with Cu deficiency and reduced with excess Cu (FIG. 5A). To test the importance of CCH in the Cu deficiency, we examined the phenotype of the cch mutant and CCH-overexpressing lines in both a wild-type and cch background. Arabidopsis transgenic plants overexpressing the CCH gene were generated in a wild-type and cch mutant background (Wt-CCH and cch-CCH, respectively). FIG. 10A shows the accumulation of CCH protein in selected transgenic lines of Wt-CCH and cch-CCH. The cch mutant and CCH-overexpressing lines showed no obvious changes in phenotype with Cu deficiency and excess Cu (FIG. 10B, data not shown). Interestingly, the atx1 mutant and ATX1-overexpressing lines showed a phenotype under Cu-deficient conditions. The atx1 and cchatx1 mutants were more sensitive to Cu deficiency, whereas ATX1-overexpressing lines were more tolerant of Cu deficiency (FIG. 10C). With Cu deficiency, the biomass and root length of ATX1-overexpressing lines were about 170% and 120%, respectively, that of the wild type (FIG. 10D, 10E). Thus, ATX1 is required for tolerance to Cu deficiency. This finding implies that ATX1 increases Cu use efficiency, which results in enhanced growth on half-strength MS media, considered a Cu-insufficient condition.

2.8 MXCXXC Motif is Required for the Function of ATX1

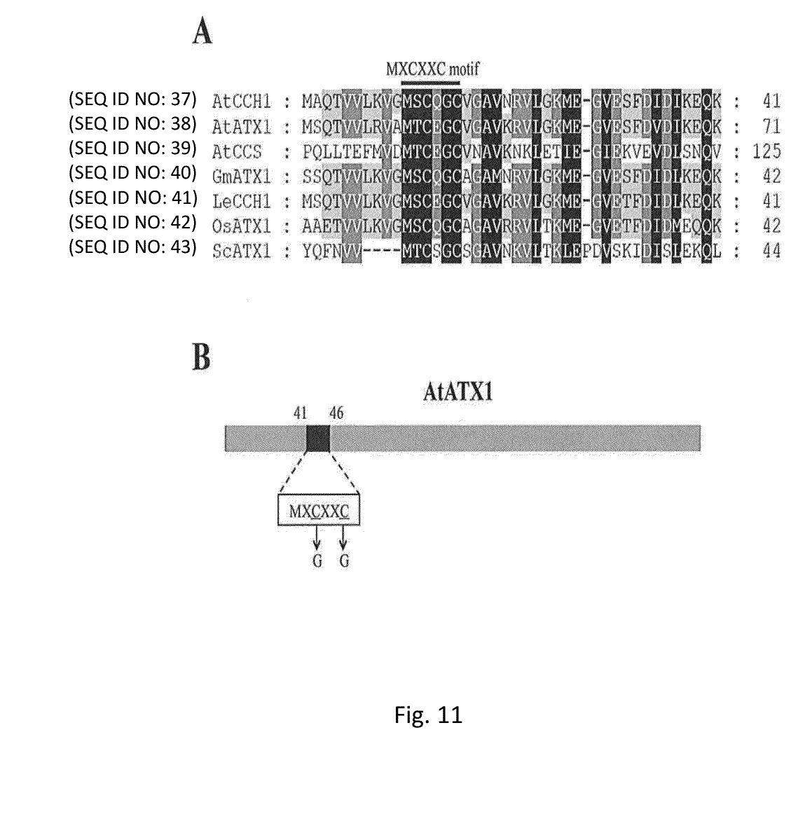

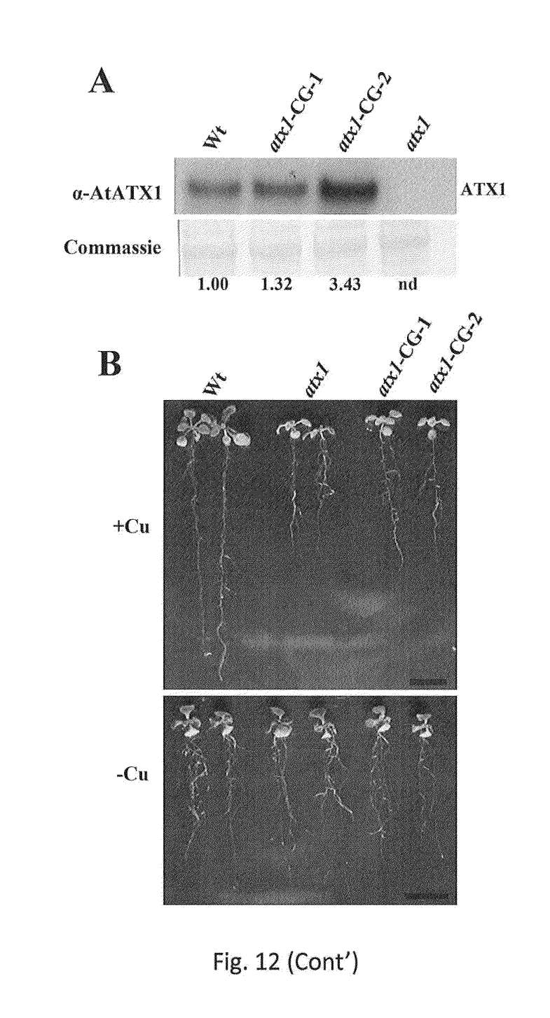

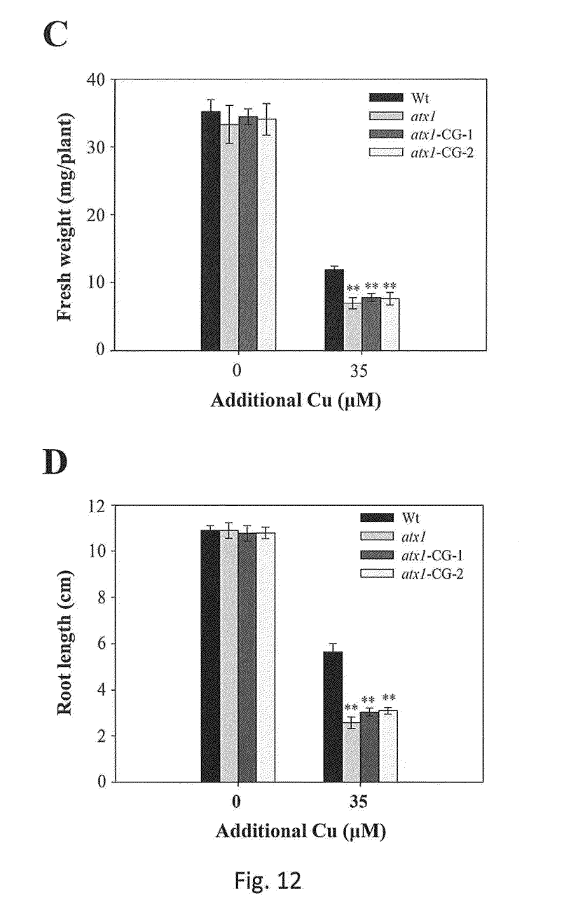

To elucidate whether the only conserved MXCXXC Cu-binding motif of ATX1 is essential for the function of ATX1 (FIG. 11), we mutated the 2 cysteine residues to glycine residues in the motif to create MXGXXG in mutated ATX1 for producing overexpressing lines in an atx1 background (atx1-CG). We detected mutated ATX1 protein accumulated in the 2 independent atx1-CG lines (FIG. 12A) but observed no rescued phenotype under Cu-excess or -deficient conditions in both lines (FIG. 12B). Sensitivity to excess Cu was similar for the atx1-CG-1 and atx1-CG-2 transgenic lines and the atx1 mutant (FIG. 12B). With excess Cu, the biomass and root length for atx1, atx1-CG-1 and -2 was about 60% and 50%, respectively, that of the wild type (FIGS. 12C and 12D). Therefore, ATX1-mediated tolerance to excess Cu may have depended on the MXCXXC motif. Furthermore, the atx1-CG-1 and atx1-CG-2 transgenic lines, similar to atx1, showed loss of tolerance to Cu deficiency (FIG. 12B). Thus, the MXCXXC Cu binding motif is required for ATX1 function in response to both excess Cu and Cu deficiency. As well, Cu chelating is the crucial action of ATX1 in conducting its biological function.

2.9 ATX1 Overexpression Enhances Cu Accumulation

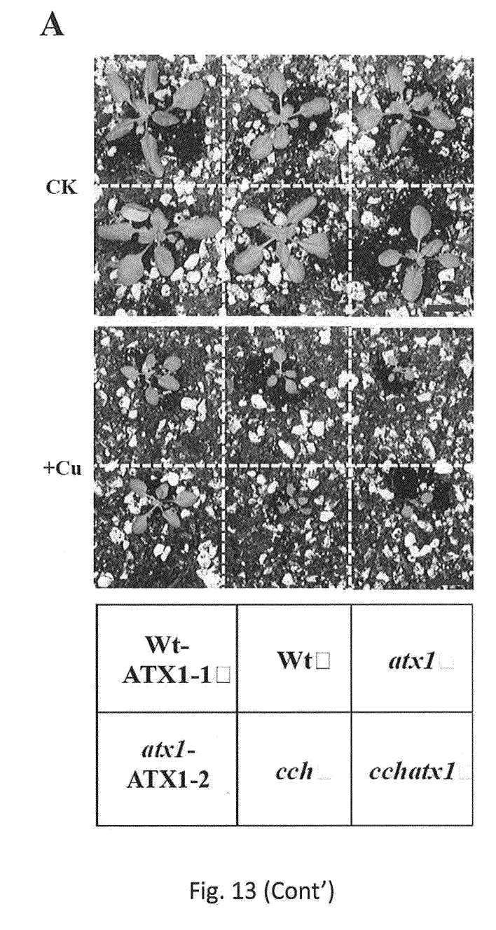

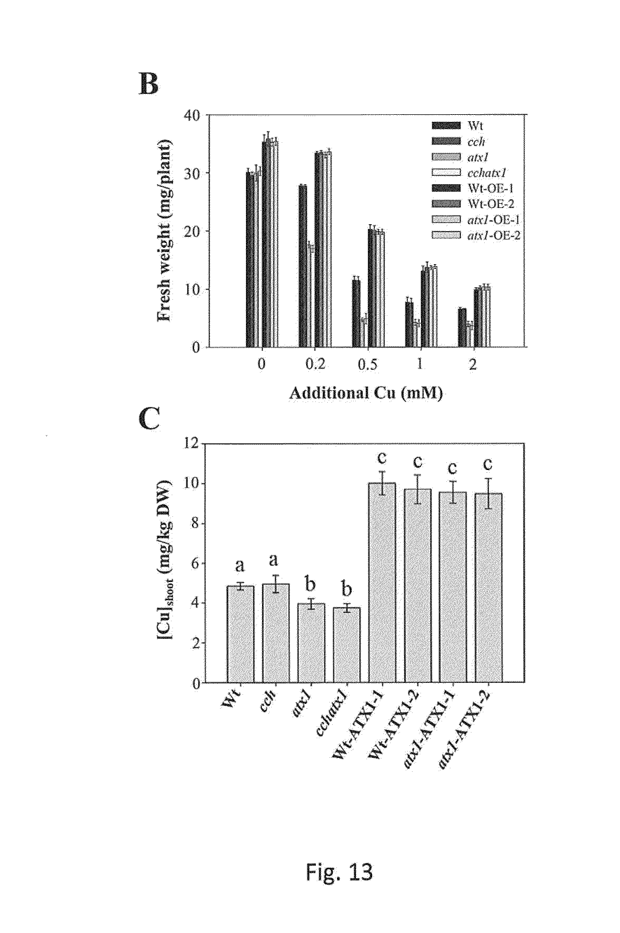

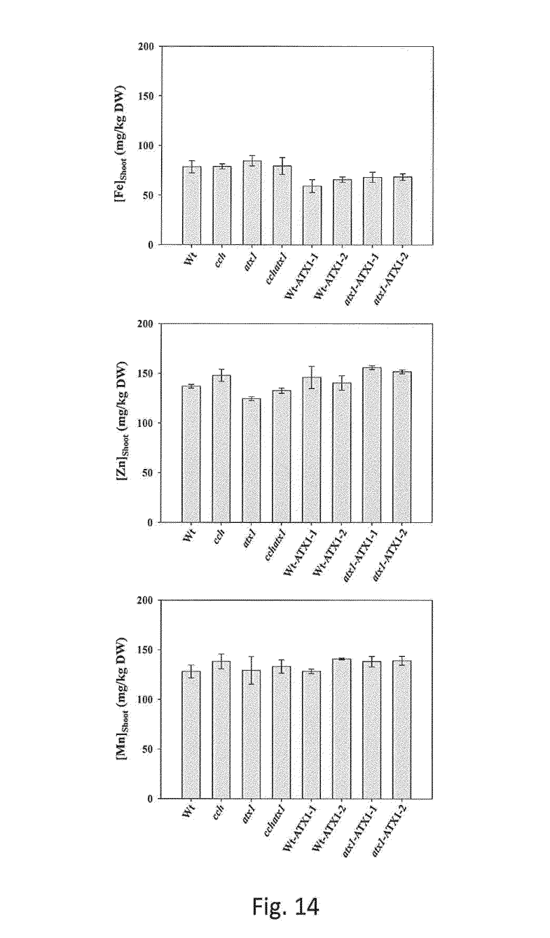

Our finding of the overexpression of ATX1 enhancing Cu tolerance implies the potential use of ATX1 for phytoremediation in Cu-contaminated soil. To mimic the natural condition, we challenged plants with Cu-grouted soil. Grouting continuously with excess Cu elevates Cu stress in soil to explicit Cu sensitive phenotype. ATX1-overexpression lines showed high Cu tolerance as compared with the wild type (FIG. 13A). The relative fresh weight was higher (170% to 180% increase) for ATX1-overexpression lines in both the wild-type or atx1 background than the wild type and was higher (320% to 340% increase) than for the atx1 and cchatx1 mutants in Cu-grouted soil (FIG. 13B). Although shoot Cu accumulation was similar for the media-grown wild type and atx1 mutant (FIG. 4), to further investigate the ATX1 function in Cu accumulation, we analyzed Cu content in these transgenic plants grown in high Cu content soil. After sowing in high Cu soil, plants were grouted with water only with reduced the influence of growth defect in high Cu toxicity. The Cu concentration was surprisingly higher, by about 200%, in shoots of Wt-ATX1-1, 2 and atx1-ATX1-1, 2 lines than in shoots of the wild type and mutants (FIG. 13C). By contrast, atx1 and cchatx1 mutants accumulated less Cu (80%) under excess Cu in soil (FIG. 13C). However, the contents of Fe, Zn and Mn remained unchanged (FIG. 14). These data again support that ATX1 plays an important role in Cu tolerance and accumulation in planta.

The overexpression of ATX1 enhances Cu accumulation and elevates the tolerance threshold to Cu toxicity. By multiplying the effects on biomass and accumulation, overexpressing ATX1 enhances Cu extraction by about 400% of the wild-type extraction. Therefore, overexpression of ATX1 leads to overaccumulation of Cu, then tolerance to excess Cu.

2.10 N-Terminal of ATX1 is Important for its Function on the Tolerance to Both Excess and Deficient Cu Conditions

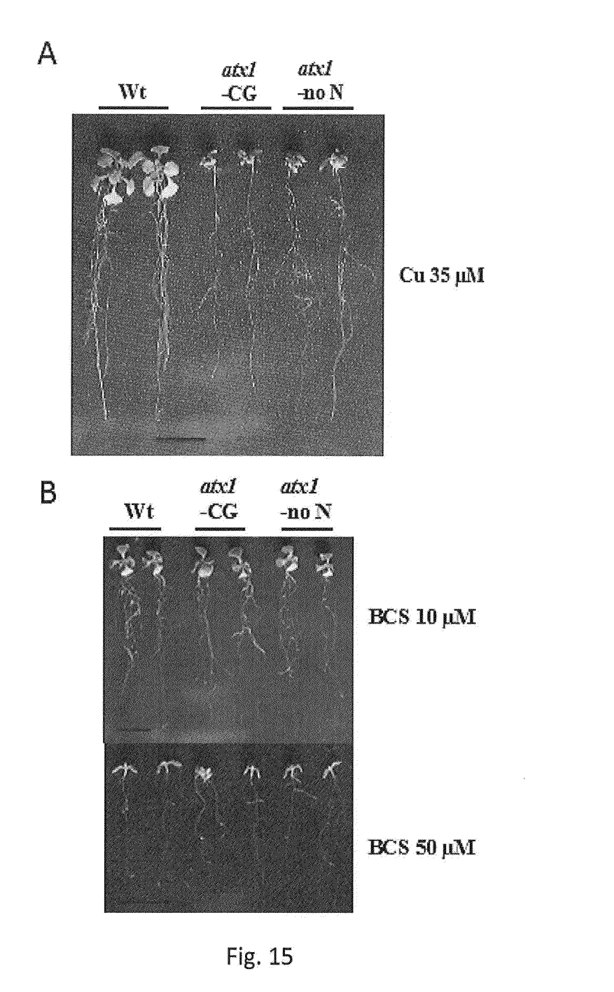

In order to examine the role of unique N-terminus in AtATX1, we overexpressed N-terminal (1-30 amino acids) deleted version of ATX1 in the atx1 mutant designated “atx1-no N”. Like atx1-CG (FIG. 12B), “atx1-no N” show neither excess Cu tolerance nor does Cu deficient resistance (FIGS. 15 A and B). Therefore, the unique N-terminus is require for ATX1 function.

3. Discussion

The homeostasis of metal ions, including macro- and micronutrients, is regulated by mechanisms of uptake, compartmentalization and translocation to support plant growth and development. Cu is one of the least-abundant micronutrients and is essential for many biochemical reactions in plant tissues (Marschner, 1995; Burkhead et al., 2009). An amount of 6 ppm Cu was considered an adequate concentration, and ≧20 ppm can induce toxicity in shoot tissues (Marschner, 1995; Burkhead et al., 2009). To prevent Cu deficiency or excess, homeostasis of Cu must be strictly fine-tuned as compared with that of other metals. Cu chaperones were thought to perform the fine tuning by the deduced dual functions of Cu trafficking and detoxification (Harrison et al., 1999). Despite the hypothetical functions of Cu chaperones, little is known about their physiological significance in plants.

In this study, we found that ATX1 but not CCH chaperones are required for tolerance to Cu excess and deficiency in Arabidopsis, which suggests that the 2 chaperones possess different homeostatic properties and distinct functions in planta. The atx1 but not cch mutant showed increased Cu sensitivity. The phenotype of the cchatx1 double mutant was similar to that of atx1 (FIG. 2C). Thus, we demonstrate the importance of ATX1 in homeostasis for tolerance to excess Cu and its induced expression by excess Cu also supports a role in Cu tolerance (FIG. 5).

Yeast ATX1 was reported to chelate Cu with excellent affinity (Pufahl et al., 1997; Shoshan and Tshuva, 2011). As well, the MXCXXC motif of yeast ATX1 acts as a high-affinity Cu binding site and is important for Cu-dependent protein-protein interaction (Pufahl et al., 1997; Shoshan and Tshuva, 2011). Alignment of protein sequences revealed that ATX1 in Arabidopsis contains only one MXCXXC motif and the only known metal binding motif (FIG. 11). We showed that this motif is required for ATX1 function. CG-ATX1 containing a mutated MXCXXC motif with 2 Cys residues replaced by 2 Gly residues could neither rescue Cu hypersensitivity nor enhance tolerance to Cu deficiency (FIG. 12). As well, transgenic lines with different CG-ATX1 levels showed complete loss of function of both excess Cu and Cu deficiency but no dominant-negative effect or intermediate phenotype. These data clearly demonstrate the specific role of the MXCXXC motif in the biological function of ATX1. Together with previous results (Pufahl et al., 1997; Hara et al., 2010), our results show that the biological function of ATX1 requires Cu chelation on the MXCXXC motif. Although CCH also possesses an MXCXXC motif, we did not observe the phenotype in the knockout mutant cch or overexpression lines under the conditions we tested. The CCH function could be compensated by redundancy of the genome's other metal-binding proteins, whose functions are currently not known (Hara et al., 2010; Shoshan and Tshuva, 2011).

Metallothioneins (MTs) are proteins of low molecular mass (4-14 kD) with rich cysteine (Cys, C) residues that chelate Cu, Zn, and Cd via Cys residues by forming sulfhydryl ligands (Hara et al., 2010). The arrangement of Cys residues is crucial in determining the metal-binding properties of MT proteins and their functions (Guo et al., 2008). Cys residues in MTs are arranged in metal-binding motifs, C—C, C—X—C, or C—X—X—C. These defined protein motifs explain MTs conferring tolerance to excess Cu, Zn and Cd. All MTs possess different affinity to various metals. For example, most MTs can bind to Cu effectively, and type 4 MTs have high affinity to Zn (Guo et al., 2008). By contrast, ATX1 contains one MXCXXC motif but no C—C, C—X—C, or C—X—X—C motifs. Therefore, ATX1 more effectively and specifically binds Cu than other metals (Badarau and Dennison, 2011). The difference in the composition of metal motifs implies that MTs and ATX1 function differentially. However, ATX1 is specifically involved in Cu homeostasis in plants. This hypothesis is further supported by our finding of Cu-specific tolerance and accumulation in ATX1-overexpressing plants and Cu-specific hypersensitivity in the atx1 mutant (FIG. 2, FIG. 6, FIG. 7, FIG. 9, FIG. 13).

In addition, the regulation of MT expression is important in tolerance to Cu toxicity (Cobbett and Goldsbrough, 2002). MTs are deduced to mobilize metal ions from senescing leaves and sequester excess metal ions (Guo, 2003). However, ATX1 and MTs differ in that expression patterns of MTs in Arabidopsis are tissue specific (Cobbett and Goldsbrough, 2002), whereas ATX1 is ubiquitously expressed in many Arabidopsis vegetative tissues (Puig et al., 2007). MTs also show redundancy in tissues. The Arabidopsis mt1a-2mt2b-1 double mutants are not sensitive to excess Cu (Guo et al., 2008), but the Arabidopsis mt1a-2mt2b-1cad1-3 triple mutant is sensitive to excess Cu (Guo et al., 2008). Therefore, MTs involved in Cu tolerance require a synergy with phytochelatin. By contrast, we found the ATX1-defective mutants atx1 and cchatx1 sensitive to excess Cu (FIG. 2). Thus, ATX1 expression may be a first-line response against excess Cu stress. ATX1 could be primarily responsible for tolerance to excess Cu, then MTs could be responsible for the escaped Cu and the process of Cu redistribution and detoxification (Guo et al., 2008).

Previous studies indicated that the transcription factor SQUAMOSA Promoter Binding Protein-Like7 (SPL7) was essential in the response to Cu deficiency (Yamasaki et al., 2009). The spl7 mutant was hypersensitive to Cu deficiency, but the expression of ATX1 was not affected in the mutant (Yamasaki et al., 2009). Therefore, the roles of SPL7 and ATX1 in Cu deficiency are independent.

The expression of ATX1 is universal and the accumulation of CCH is mostly in phloem-enucleated sieve elements (Mira et al., 2001; Puig et al., 2007). The expression of CCH is induced by Cu deficiency and that of ATX1 increases under excess Cu, which again supports the hypothesis of differential functions between ATX1 and CCH (FIG. 5). Furthermore, the unique C-terminal domain of CCH blocks the interaction of RAN1 and HMA5 (Andres-Colas et al., 2006; Puig et al., 2007). These observations suggest that CCH has a specific function that differs from that of ATX1 regulated by its unique C-terminal domain.

Yeast two-hybrid screening revealed that 2 transporters, RAN1 and HMA5, interact with ATX1 (Andres-Colas et al., 2006; Puig et al., 2007), which may suggest the Cu delivery role of ATX1. The phenotype of ran1 can be suppressed by additional Cu supply, but it is not Cu hypersensitive. We did not observe any deficiency in ethylene-related responses in the atx1 mutant. Arabidopsis may have alternative pathways to compensate ATX1 function in the ethylene response.

The closest homolog of RAN1 in Arabidopsis is HMA5 (Williams and Mills, 2005). HMA5 is an efflux transporter of Cu. The expression of HMA5 is induced by Cu and is mainly in roots and flowers (Andres-Colas et al., 2006). The hma5 mutant is Cu hypersensitive in the root and is accompanied by a wave-like root growth. Therefore, HMA5 was proposed to have a role in Cu translocation from root to shoot (Andres-Colas et al., 2006). On the basis of the interaction between ATX1 and HMA5, ATX1 was proposed to deliver Cu to HMA5 for Cu detoxification in roots and translocation to shoots. We observed root hypersensitivity and high expression of HMA5 (FIG. 8) with low shoot Cu accumulation in the atx1 mutant (FIG. 13C), which supports ATX1 involved in Cu detoxification with HMA5. In addition, ATX1 also expresses in the shoot and atx1 shows hypersensitivity in the shoot, which suggests its additional role in the shoot. Although only RAN1 and HMA5 have been found to interact with ATX1, ATX1 may also interact with other proteins, at least in the shoot, for Cu homeostasis. Besides, the universal expression of ATX1 was suggested (Puig et al., 2007), but the tissue/organ-specific expression had not been clarified under various Cu conditions. It is worthy for further studies to elucidate the detail mechanism in different tissues.

Cu chaperone mutants and the wild type showed similar growth under half-strength MS media. However, the atx1 mutant showed sensitivity to both excess Cu and Cu deficiency, whereas ATX1 overexpression conferred tolerance to excess Cu and Cu deficiency (FIG. 9 and FIG. 10). ATX1 may be involved in chelating Cu under Cu overload and facilitate Cu usage under deficiency. Recently, the tonoplast Cu transporter COPTS was shown to act as an exporter and was required for tolerance to Cu deficiency; COPTS may transport Cu from the vacuole or prevacuolar compartment to the cytosol to redistribute Cu in cells during Cu deficiency (Garcia-Molina et al., 2011; Klaumann et al., 2011; Pilon, 2011). ATX1 may have a role in adapting Cu released from the vacuole via COPTS for use under Cu deficiency.

Although half-strength MS media is a Cu-sufficient condition, growth media with about 3 to 5 μM Cu is considered abundant that makes better vegetative growth than in half-strength MS media (Yamasaki et al., 2009; Kopittke et al., 2010). Our finding that the wild type grew best in half-strength MS with 5 μM CuSO₄ (FIG. 2D) supports previous observations and explains the enhanced growth of ATX-overexpressing lines with half-strength MS. Therefore, ATX1 overexpression increases growth fitness under Cu-deficient and -excess conditions by facilitating Cu usage and arresting unchelated Cu from causing toxicity, respectively. It is worth mentioning here that low Cu condition could be more biologically relevant. Reduced growth was observed in the atx1 and cchatx1 mutants under Cu deficient treatment.

This indicates that Cu deficiency imposes a positive selection advantage on ATX1.

### 4. Conclusions

In summary, we demonstrate the biological function of ATX1 in Arabidopsis in response to excess and deficient Cu. ATX1 contributes to tolerance to excess Cu and tolerance to Cu deficiency. Its function requires the Cu-binding MXCXXC motif and the N-terminal domain. The present invention thus can apply a transgenic plant overexpressing a ATX1-like polypeptide with the necessary Cu-binding MXCXXC motif and the N-terminal domain in phytoremediation to remove copper contamination.

### Sequence Information

```
>Arabidopsis thaliana-ATX1 protein sequence
(SEQ ID NO: 1)
MLKDLFQAVSYQNTASLSLFQALSVVESKAMSQTVVLRVAMTCEGCVGAV
KRVLGKMEGVESFDVDIKEQKVIVKGNVQPDAVLQTVIKTGKKTAFWEAE
GETAKA
>Arabidopsis thaliana-ATX1 N-terminal sequence
(SEQ ID NO: 2)
MLKDLFQAVSYQNTASLSLFQALSVVESKA
>ATX1-like polypeptide C-terminal copper binding
motif
(SEQ ID NO: 3)
MXCXXC
>ATX1-like polypeptide C-terminal motif
(SEQ ID NO: 4)
MSQTVVLRVAMTCEGCVGAVKRVLGKMEGVESFDVDIKEQKVTVKGNVQPDAVLQTVTK
  AE T K G S Q A N E LQ Y INLQKK V I TSE FKA S
          E K S S Q D ME N EK K
                  E K
TGKKTXFWXXXXXXXXX
S RP Y
      A
(thin-underlined residues mean conserved, thick-underlined
residues mean changeable, X means any amino acid residues)
>ATX1-like polypeptide C-terminal motif
(SEQ ID NO: 5)
XXXXXXXXVXMXCXGCXGAVXXVLXKXXXXVXXXXXXXXXXXVXVXXXXXXXX
XXXXXXKXXXXXXXXXXXXXXXXX
(underlined residues mean conserved, X means any
amino acid residues)
>Arabidopsis thaliana-ATX1 protein, residues 31-106
(SEQ ID NO: 6)
MSQTVVLRVAMTCEGCVGAVKRVLGKMEGVESFDVDIKEQKVTVKGNVQPDAVLQTV
TKTGKKTAFWEAEGETAKA
>Hevea brasiliensis_gi|290886187|_ATX1-L 87% identity
(SEQ ID NO: 7)
MSQTVVLKVGMSCEGCVGAVKRVLGKMEGVESYDIDLKEQKVTVKGNVQPEAVLQTV
SKTGKKTTFWEAEAPAEPETKPAETVTVA
>Jatropha curcas_gi|257219554|_ATX1-L 81% identity
(SEQ ID NO: 8)
MSQTVVLKVGMSCQGCVGAVKRVLGKMEGVESYDIDLQEQKVTVKGNVQPEAVLQTV
SKTGKKTEFWEAEAPAAPETKPAETVSEPAETVAVA
>Populus glandulosa_gi|47176684|_ATX1_L 85% identify
(SEQ ID NO: 9)
MSQTVVLKVGMSCEGCVGAVKRVLGKMEGVESYDIDLKEQKVTVKGNVQPDAVLQTV
SKTGKKTAFWEAEAPAEPAKPAETVAAA
>Populus trichocarpa_gi|118481259|_ATX1 82% identify
(SEQ ID NO: 10)
MSQTVVLKVGMSCGGCVGAVKRVLGKMEGVESYDIDLKEQKVTVKGNVQPDAVLQTV
SKTGKKTTFWEAEAPAEPATAETLAAA
>Zea mays_gi|226491116|_ATX1 83% identify
(SEQ ID NO: 11)
MAQTVVLKVGMSCEGCVGAVKRVLGKMEGVESYDVDIMEQKVTVKGNVTPDAVLQTV
SKTGKKTSFWEAEAVTSESATPAGATA
>Populus trichocarpa_gi|224110212|_CCH 82% identify
(SEQ ID NO: 12)
QTVVLKVGMSCEGCVGAVKRVLGKMEGVESYDIDLKEQKVTVKGNVQPDAVLQTVSK
TGKKTAFWEAEAPAE
>Oryza sativa Japonica Group_gi|115475275|_ATX1_L 82% identify
(SEQ ID NO: 13)
MAETVVLRVGMSCEGCVGAVKRVLGKMQGVESFDVDIKEQKVTVKGNVTPDAVLQTV
SKTGKKTSFWDAEPAPVEATAASS
>Medicago truncatula_gi|357442955|_ATX1 66% identify
(SEQ ID NO: 14)
MSSQTVTLKVGMSCEGCVGAVKRVLGKLDGVESYDIDLKEQKVVVKGNVEPDTVLKT
VSKTGKPTAFWEAEAPSETKAQ
>Arabidopsis thaliana_gi|15228869|_CCH 77% identify
(SEQ ID NO: 15)
MAQTVVLKVGMSCQGCVGAVNRVLGKMEGVESFDIDIKEQKVTVKGNVEPEAVFQTV
SKTGKKTSYWPVEAEAEPKAEADPKVETVTETKTEAETKTEAKVDAKADVEPKAAEA
ETKPSQV
>Thellungiella halophila_gi|312282829|_ATX1 75% identify
(SEQ ID NO: 16)
MSQTVVLKVGMSCQGCVGAVNRVLGKMEGVESFDIDIKEQKVTVKGNVEPEAVFQTV
SKTGKKTSYWPVDAEAEPKAEAEPKKETETETKTEAETKTEAKVDVEPKLAEAESKP
SQV
>Solanum lycopersicum_gi|460409110|_ATX1L 74% identify
(SEQ ID NO: 17)
MSQTVVLKVGMSCQGCVGAVNRVLGKMEGVESFDIDIKEQKVTVIGNVEPEAVFQTV
SKTGKKTSYWEEPAPASAPEPETKPVEEKPVEEKPTETPAEPEPKPTEEKPAETVA
>Glycine max_gi|351724867|_ATX1 71% identify
(SEQ ID NO: 18)
MSSQTVVLKVGMSCQGCAGAVNRVLEKMEGVESFDIDLKEQKVTVKGNVQP
DEVLQAVSKSGKKTAFWVDEAQPPENKPSETAPVTSAENDNKASESGPVASE
NKPPEAAHVASADPETKPSETAVETVA
>Glycine max_gi|255637332|_CCH 71% identify
(SEQ ID NO: 19)
MSSQTVVLKVGMSCQGCAGAVNRVLGKMEGVESFDIDLKEQKVTVKGNVESDEVLQA
VSKSGKKTAFWVDEAPQSKNKPLESAPVASENKPSEAATVASAEPENKPSEAAIVDS
AEPENKPSDTVVETVA
>Medicago truncatula_gi|217072900|_CCH 66% identify
(SEQ ID NO: 20)
MSSETVVLKVKMSCSGCSGAVNRVLEKMEGVESFDIDMKEQKVTVKGNVKPQDVFDT
VSKTGKKTEFWVEPENNPTETATEAEPENKPSEAVTIDPVEPDNKPSETATVVSIEP
ENKPSETATVAA
>Plantago major_gi|53748477|_ATX1 74% identify
(SEQ ID NO: 21)
MSQTVELKVGMSCQGCVGAVKRVLGKMEGVESFDIDIEKQKVTVKGNVEKEAVLQTV
SKTGKKTEFWPEEAAEPEAKITEAPAPVEAKPTEAPAAEPESKPTEAVVTA
>Plantago major_gi|53748477|_CCH 74% identify
(SEQ ID NO: 22)
MSQTVELKVGMSCQGCVGAVKRVLGKMEGVESFDIDIEKQKVTVKGNVEKEAVLQTV
SKTGKKTEFWPEEAAEPEAKITEAPAPVPEAKPTEAPAAEPESKPTEAVVTA
>Knorringia sibirica_gi|186926670|_ATX1_L, 68% identify
(SEQ ID NO: 23)
MSQTVVLKVEMTCQGCVGAVQRVLGKMEGVESFDVNLEEKKVTVNGNVDPEAVLQKV
SKTGRATSFWDESAPPSA
>Rheum australe_gi|197312871|_ATX1_L 72% identify
(SEQ ID NO: 24)
MSQTVVLKVEMTCQGCVGAVQRVLGKMEGVESFNVDLKEKKVTVNGNVDPEAVLQKV
SKTGKKTSFWDEAAPSSA
>Saccharomyces cerevisiae_gi|190409232|_ATX1 32% identify
(SEQ ID NO: 25)
MAEIKHYQFNVVMTCSGCSGAVNKVLTKLEPDVSKIDISLEKQLVDVYTTLPYDFIL
EKIKKTGKEVRSGYQL
```

### REFERENCES

Andres-Colas N, Sancenon V, Rodriguez-Navarro S, Mayo S, Thiele D J, Ecker J R, Puig S, Penarrubia L (2006) The Arabidopsis heavy metal P-type ATPase HMA5 interacts with metallochaperones and functions in copper detoxification of roots. Plant J 45: 225-236
Badarau A, Dennison C (2011) Thermodynamics of copper and zinc distribution in the cyanobacterium Synechocystis PCC 6803. Proc Natl Acad Sci USA 108: 13007-13012
Brewer G J (2010) Copper toxicity in the general population. Clin Neurophysiol 121: 459-460
Burkhead J L, Reynolds K A, Abdel-Ghany S E, Cohu C M, Pilon M (2009) Copper homeostasis. New Phytol 182: 799-816
Casareno R L, Waggoner D, Gitlin J D (1998) The copper chaperone CCS directly interacts with copper/zinc superoxide dismutase. J Biol Chem 273: 23625-23628
Cho U H, Sohn J Y (2004) Cadmium-induced changes in antioxidative systems, hydrogen peroxide content, and lipid peroxidation in Arabidopsis thaliana. Journal of Plant Biology 47: 262-269
Chu C C, Lee W C, Guo W Y, Pan S M, Chen L J, Li H M, Jinn T L (2005) A copper chaperone for superoxide dismutase that confers three types of copper/zinc superoxide dismutase activity in Arabidopsis. Plant Physiol 139: 425-436
Cobbett C, Goldsbrough P (2002) Phytochelatins and metallothioneins: roles in heavy metal detoxification and homeostasis. Annu Rev Plant Biol 53: 159-182
Drazkiewicz M, Skorzynska-Polit E, Krupa Z (2004) Copper-induced oxidative stress and antioxidant defence in Arabidopsis thaliana. Biometals 17: 379-387
Garcia-Molina A, Andres-Colas N, Perea-Garcia A, Del Valle-Tascon S, Penarrubia L, Puig S (2011) The intracellular Arabidopsis COPTS transport protein is required for photosynthetic electron transport under severe copper deficiency. Plant J 65: 848-860
Guo W J, Meetam M, Goldsbrough P B (2008) Examining the specific contributions of individual Arabidopsis metallothioneins to copper distribution and metal tolerance. Plant Physiol 146: 1697-1706
Guo W J, Weenun Bundithya, Peter B. Goldsbrough (2003) Characterization of the Arabidopsis metallothionein gene family: tissue-specific expression and induction during senescence and in response to copper. New Phytologist 159: 369-381
Hara M, Kashima D, Horiike T, Kuboi T (2010) Metal-binding characteristics of the protein which shows the highest histidine content in the Arabidopsis genome. Plant Biotechnology 27: 475-480
Harrison M D, Jones C E, Dameron C T (1999) Copper chaperones: function, structure and copper-binding properties. Journal of Biological Inorganic Chemistry 4: 145-153
Himelblau E, Mira H, Lin S J, Culotta V C, Penarrubia L, Amasino R M (1998) Identification of a functional homolog of the yeast copper homeostasis gene ATX1 from Arabidopsis. Plant Physiol 117: 1227-1234
Hirayama T, Kieber J J, Hirayama N, Kogan M, Guzman P, Nourizadeh S, Alonso J M, Dailey W P, Dancis A, Ecker J R (1999) RESPONSIVE-TO-ANTAGONIST1, a Menkes/Wilson disease-related copper transporter, is required for ethylene signaling in Arabidopsis. Cell 97: 383-393
Kampfenkel K, Kushnir S, Babiychuk E, Inze D, Van Montagu M (1995) Molecular characterization of a putative Arabidopsis thaliana copper transporter and its yeast homologue. J Biol Chem 270: 28479-28486
Klaumann S, Nickolaus S D, Furst S H, Starck S, Schneider S, Ekkehard Neuhaus H, Trentmann O (2011) The tonoplast copper transporter COPTS acts as an exporter and is required for interorgan allocation of copper in Arabidopsis thaliana. New Phytol 192: 393-404
Kopittke P M, Blarney F P, Asher C J, Menzies N W (2010) Trace metal phytotoxicity in solution culture: a review. J Exp Bot 61: 945-954
Kupper H, Setlik I, Setlikova E, Ferimazova N, Spiller M, Kupper F C (2003) Copper-induced inhibition of photosynthesis: limiting steps of in vivo copper chlorophyll formation in Scenedesmus quadricauda. Functional Plant Biology 30: 1187-1196
Lequeux H, Hermans C, Lutts S, Verbruggen N (2010) Response to copper excess in Arabidopsis thaliana: Impact on the root system architecture, hormone distribution, lignin accumulation and mineral profile. Plant Physiol Biochem 48: 673-682
Lin S J, Culotta V C (1995) The ATX1 gene of Saccharomyces cerevisiae encodes a small metal homeostasis factor that protects cells against reactive oxygen toxicity. Proc Natl Acad Sci USA 92: 3784-3788
Lin Y F, Liang H M, Yang S Y, Boch A, Clemens S, Chen C C, Wu J F, Huang J L, Yeh KC (2009) Arabidopsis IRT3 is a zinc-regulated and plasma membrane localized zinc/iron transporter. New Phytol 182: 392-404
Liu H C, Liao H T, Charng Y Y (2011) The role of class A1 heat shock factors (HSFA1s) in response to heat and other stresses in Arabidopsis. Plant Cell Environ 34: 738-751
Marschner H (1995) Mineral Nutrition of Higher Plants, Ed second. Academic Press, London
Mira H, Martinez-Garcia F, Penarrubia L (2001) Evidence for the plant-specific intercellular transport of the Arabidopsis copper chaperone CCH. Plant J 25: 521-528
Mira H, Vilar M, Perez-Paya E, Penarrubia L (2001) Functional and conformational properties of the exclusive C-domain from the Arabidopsis copper chaperone (CCH). Biochem J 357: 545-549
Patsikka E, Kairavuo M, Sersen F, Aro E M, Tyystjarvi E (2002) Excess copper predisposes photosystem II to photoinhibition in vivo by outcompeting iron and causing decrease in leaf chlorophyll. Plant Physiol 129: 1359-1367
Pilon M (2011) Moving copper in plants. New Phytol 192: 305-307
Pufahl R A, Singer C P, Peariso K L, Lin S J, Schmidt P J, Fahrni C J, Culotta V C, Penner-Hahn J E, O′Halloran T V (1997) Metal ion chaperone function of the soluble Cu(I) receptor Atx1. Science 278: 853-856
Puig S, Andres-Colas N, Garcia-Molina A, Penarrubia L (2007) Copper and iron homeostasis in Arabidopsis: responses to metal deficiencies, interactions and biotechnological applications. Plant Cell Environ 30: 271-290
Puig S, Mira H, Dorcey E, Sancenon V, Andres-Colas N, Garcia-Molina A, Burkhead J L, Gogolin K A, Abdel-Ghany S E, Thiele D J, Ecker J R, Pilon M, Penarrubia L (2007) Higher plants possess two different types of ATX1-like copper chaperones. Biochem Biophys Res Commun 354: 385-390
Puig S, Thiele DJ (2002) Molecular mechanisms of copper uptake and distribution. Curr Opin Chem Biol 6: 171-180
Rae T D, Schmidt P J, Pufahl R A, Culotta V C, O′Halloran T V (1999) Undetectable intracellular free copper: the requirement of a copper chaperone for superoxide dismutase. Science 284: 805-808
Sancenon V, Puig S, Mateu-Andres I, Dorcey E, Thiele D J, Penarrubia L (2004) The Arabidopsis copper transporter COPT1 functions in root elongation and pollen development. J Biol Chem 279: 15348-15355
Sancenon V, Puig S, Mira H, Thiele D J, Penarrubia L (2003) Identification of a copper transporter family in Arabidopsis thaliana. Plant Mol Biol 51: 577-587
Schutzendubel A, Polle A (2002) Plant responses to abiotic stresses: heavy metal-induced oxidative stress and protection by mycorrhization. J Exp Bot 53: 1351-1365
Shibasaki K, Uemura M, Tsurumi S, Rahman A (2009) Auxin response in Arabidopsis under cold stress: underlying molecular mechanisms. Plant Cell 21: 3823-3838
Shoshan M S, Tshuva E Y (2011) The MXCXXC class of metallochaperone proteins: model studies. Chem Soc Rev 40: 5282-5292
Skorzynska-Polit E, Drazkiewicz M, Krupa Z (2010) Lipid peroxidation and antioxidative response in Arabidopsis thaliana exposed to cadmium and copper. Acta Physiologiae Plantarum 32: 169-175
Sun B Y, Kan S H, Zhang Y Z, Deng S H, Wu J, Yuan H, Qi H, Yang G, Li L, Zhang X H, Xiao H, Wang Y J, Peng H, Li Y W (2010) Certain antioxidant enzymes and lipid peroxidation of radish (Raphanus sativus L.) as early warning biomarkers of soil copper exposure. J Hazard Mater 183: 833-838
Williams L E, Mills R F (2005) P(1B)-ATPases—an ancient family of transition metal pumps with diverse functions in plants. Trends Plant Sci 10: 491-502
Woeste K E, Kieber J J (2000) A strong loss-of-function mutation in RAN1 results in constitutive activation of the ethylene response pathway as well as a rosette-lethal phenotype. Plant Cell 12: 443-455
Yamasaki H, Hayashi M, Fukazawa M, Kobayashi Y, Shikanai T (2009) SQUAMOSA Promoter Binding Protein-Like7 Is a Central Regulator for Copper Homeostasis in Arabidopsis. Plant Cell 21: 347-361

What is claimed is:

1. A transgenic plant transformed with a recombinant polynucleotide comprising a nucleotide sequence encoding an antioxidant protein 1 (ATX1)-like polypeptide, operatively linked to an expression control sequence, wherein the ATX-like polypeptide has an amino acid sequence having at least 30%, 35%, 40%, 45%, 50%, 55%, 60%, 65%, 70%, 75%, 80%, 85%, 90%, or 95% identity to SEQ ID NO: 1 and exhibits a copper binding activity, in which the amino acid sequence of the ATX-like polypeptide has (i) an N-terminal sequence having at least 60%, 65%, 70%, 75%, 80%, 85%, 90%, or 95% identity to SEQ ID NO: 2, corresponding to positions 1 to 30 of SEQ ID NO: 1, and (ii) a C-terminal sequence comprising a copper binding motif of SEQ ID NO: 3, corresponding to positions 41 to 46 of SEQ ID NO: 1.

2. The transgenic plant of claim 1, wherein the N-terminal sequence is SEQ ID NO: 2.

3. The transgenic plant of claim 1, wherein the C-terminal sequence comprises SEQ ID NO: 4, corresponding to positions 31 to 106 of SEQ ID NO: 1.

4. The transgenic plant of claim 3, wherein the C-terminal sequence is selected from the group consisting of SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23 and SEQ ID NO: 24.

5. The transgenic plant of claim 4, wherein the C-terminal sequence is selected from the group consisting of SEQ ID NO: 6, SEQ ID NO: 7, SEQ ID NO: 8, SEQ ID NO: 9, SEQ ID NO: 10, SEQ ID NO: 11, SEQ ID NO: 12 and SEQ ID NO: 13.

6. The transgenic plant of claim 1, wherein the C-terminal sequence comprises SEQ ID NO: 5.

7. The transgenic plant of claim 6, wherein the C-terminal sequence is SEQ ID NO: 25.

9. The transgenic plant of claim 1, the ATX-like polypeptide has 90 to 250 consecutive amino acid residues in total length, wherein the N-terminal sequence covers amino acid residues 1-30 and the C-terminal sequence is fused with the N-terminal sequence, covering amino acid residues 31 to the end.

10. The transgenic plant of claim 1, the ATX-like polypeptide is composed of SEQ ID NO: 2 as the N-terminal sequence, fused with the C-terminal sequence selected from the group consisting of SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24 and SEQ ID NO: 25.

11. The transgenic plant of claim 1, wherein the ATX1-like protein has the amino acid sequence of SEQ ID NO: 1.

12. The transgenic plant of claim 1, wherein the nucleotide sequence encoding the ATX1-like protein is SEQ ID NO: 26.

13. The transgenic plant of claim 1, wherein the transgenic plant is monocotyledon or dicotyledon.

14. The transgenic plant of claim 13, wherein the monocotyledon is selected from the group consisting of rice, barley, wheat, rye, oat, corn, bamboo, sugar cane, onion, leek and ginger.

15. The transgenic plant of claim 13, wherein the dicotyledon is selected from the group consisting of Arabidopsis, eggplant, soybean, mung bean, kidney bean, pea, tobacco, lettuce, spinach, sweet potato, carrot, melon, cucumber and pumpkin.

16. The transgenic plant of claim 1, wherein the transgenic plant is resistant to excess or deficiency of copper.

17. The transgenic plant of claim 1, wherein the transgenic plant accumulates copper in a higher amount than a wild type plant of the same species while being grown under the same conditions.

18. A method for producing a transgenic plant, comprising
(a) transforming a plant cell with a recombinant polynucleotide comprising a nucleotide sequence encoding an antioxidant protein 1 (ATX1)-like polypeptide, operatively linked to an expression control sequence, wherein the ATX-like polypeptide has an amino acid sequence having at least 30%, 35%, 40%, 45%, 50%, 55%, 60%, 65%, 70%, 75%, 80%, 85%, 90%, or 95% identity to SEQ ID NO: 1 and exhibits a copper binding activity, in which the amino acid sequence of the ATX-like polypeptide has (i) an N-terminal sequence having at least 60%, 65%, 70%, 75%, 80%, 85%, 90%, or 95% identity to SEQ ID NO: 2, corresponding to positions 1 to 30 of SEQ ID NO: 1, and (ii) a C-terminal sequence comprising a copper binding motif of SEQ ID NO: 3, corresponding to positions 41 to 46 of SEQ ID NO: 1; and
(b) growing the recombinant plant cell obtained in (a) to generate a transgenic plant.

19. The method of claim 18, wherein the N-terminal sequence is SEQ ID NO: 2.

20. The method of claim 18, wherein the C-terminal sequence is selected from the group consisting of SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24 and SEQ ID NO: 25.

21. The method of claim 18, wherein the ATX1-like protein has the amino acid sequence of SEQ ID NO: 1.

22. The method of claim 18, wherein the transgenic plant is monocotyledon or dicotyledon.

23. The method of claim 22, wherein the monocotyledon is selected from the group consisting of rice, barley, wheat, rye, oat, corn, bamboo, sugar cane, onion, leek and ginger.

24. The method of claim 22, wherein the dicotyledon is selected from the group consisting of Arabidopsis, eggplant, soybean, mung bean, kidney bean, pea, tobacco, lettuce, spinach, sweet potato, carrot, melon, cucumber and pumpkin.

25. A method of phytoremediation of an environment contaminated with copper, comprising
(a) selecting an environment contaminated with copper; and
(b) growing, in said environment, a transgenic plant transformed with a recombinant polynucleotide comprising a nucleotide sequence encoding an antioxidant protein 1 (ATX1)-like polypeptide, operatively linked to an expression control sequence, wherein the ATX-like polypeptide has an amino acid sequence having at least 30%, 35%, 40%, 45%, 50%, 55%, 60%, 65%, 70%, 75%, 80%, 85%, 90%, or 95% identity to SEQ ID NO: 1 and exhibits a copper binding activity, in which the amino acid sequence of the ATX-like polypeptide has (i) an N-terminal sequence having at least 60%, 65%, 70%, 75%, 80%, 85%, 90%, or 95% identity to SEQ ID NO: 2, corresponding to positions 1 to 30 of SEQ ID NO: 1, and (ii) a C-terminal sequence comprising a copper binding motif of SEQ ID NO: 3, corresponding to positions 41 to 46 of SEQ ID NO: 1, wherein the transgenic plant accumulates and removes an amount of copper from the environment.

26. The method of claim 25, wherein the N-terminal sequence is SEQ ID NO: 2.

27. The method of claim 25, wherein the C-terminal sequence is selected from the group consisting of SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24 and SEQ ID NO: 25.

28. The method of claim 25, wherein the ATX1-like protein has the amino acid sequence of SEQ ID NO: 1.

29. The method of claim 25, wherein the transgenic plant is monocotyledon or dicotyledon.

29. The method of claim 29, wherein the monocotyledon is selected from the group consisting of rice, barley, wheat, rye, oat, corn, bamboo, sugar cane, onion, leek and ginger.

30. The method of claim 29, wherein the dicotyledon is selected from the group consisting of Arabidopsis, eggplant, soybean, mung bean, kidney bean, pea, tobacco, lettuce, spinach, sweet potato, carrot, melon, cucumber and pumpkin.

31. A method for promoting growth a plant, comprising
(a) introducing to a plant cell, a recombinant polynucleotide comprising a nucleotide sequence encoding an antioxidant protein 1 (ATX1)-like polypeptide, operatively linked to an expression control sequence, wherein the ATX-like polypeptide has an amino acid sequence having at least 30%, 35%, 40%, 45%, 50%, 55%, 60%, 65%, 70%, 75%, 80%, 85%, 90%, or 95% identity to SEQ ID NO: 1 and exhibits a copper binding activity, in which the amino acid sequence of the ATX-like polypeptide has (i) an N-terminal sequence having at least 60%, 65%, 70%, 75%, 80%, 85%, 90%, or 95% identity to SEQ ID NO: 2, corresponding to positions 1 to 30 of SEQ ID NO: 1, and (ii) a C-terminal sequence comprising a copper binding motif of SEQ ID NO: 3, corresponding to positions 41 to 46 of SEQ ID NO: 1, to obtain a transformed plant cell, and
(b) producing a transformed plant from said transformed plant, wherein the ATX1-like polypeptide is expressed in the transgenic plant at a level sufficient to promote growth of the plant.
